(19) United States
(12) Patent Application Publication
(10) Pub. No.: US 2023/0069987 A1
(43) Pub. Date: Mar. 9, 2023

(54) METHODS AND SYSTEMS FOR GENERATING BEHAVIORAL INSIGHTS USING SURVEY INSTRUMENTS AND DIABETES TREATMENT INFORMATION

(71) Applicant: Eli Lilly and Company, Indianapolis, IN (US)

(72) Inventors: Rhett Guy ALDEN, Boston, MA (US); Stephanie Smith EDWARDS, Milton, MA (US); Lawrence FISHER, Mill Valley, CA (US); Jennal Lynn JOHNSON, Indianapolis, IN (US); Danielle Marie-Hessler JONES, El Cerrito, CA (US); William Howard POLONSKY, Encinitas, CA (US); Howard Allan WOLPERT, Brookline, MA (US)

(21) Appl. No.: 17/799,316

(22) PCT Filed: Feb. 17, 2021
(86) PCT No.: PCT/US2021/018311

§ 371 Date: Aug. 12, 2022

Related U.S. Application Data
(60) Provisional application No. 62/977,773, filed on Feb. 18, 2020.

(51) Int. Cl. G16H 10/20; G16H 10/60; G16H 15/00; G16H 50/30; G16H 20/70

(52) U.S. Cl. CPC G16H 10/20; G16H 10/60; G16H 15/00; G16H 50/30; G16H 20/70

(57) ABSTRACT

A computerized method and system is provided for generating and presenting to a user behavioral insights impacting health outcomes for a person with diabetes. The method comprises scoring responses to one or more patient reported outcome (PRO) survey instruments completed by the user. The scoring generates one or more scores that measures the extent to which the person is experiencing a different social, financial, emotional, or psychological issue. The method also comprises analyzing insulin dosage and/or glucose measurement information to derive adverse health outcomes experienced by the person during a monitored time period. The generated scores and the derived adverse health outcomes are analyzed together to generate one or more behavioral insights that may impact health outcomes for the person. Each behavioral insight may comprise a correlation between one of the derived adverse health outcomes and one or more of the generated scores.

### FIELD OF THE DISCLOSURE

The present disclosure relates to systems and methods for generating and presenting behavioral insights impacting health outcomes. More particularly, the present disclosure relates to generating and presenting to a user behavioral insights that may impact health outcomes for a person with diabetes (PwDs).

### BACKGROUND OF THE DISCLOSURE

Persons with diabetes often exhibit undesirable health outcomes, such as episodes of hyperglycemia (also referred to herein as “hypers”, in which glucose levels are higher than normal or desirable) or hypoglycemia (also referred to herein as “hypos”, in which glucose levels are lower than normal or desirable). During doctor's visits, Health Care Providers (HCPs) can review quantitatively measurable metrics regarding health outcomes with their patients. For example, HCPs can review and/or discuss data such as the frequency and/or severity of hyper- or hypo-glycemic episodes, or changes in the PwD's HbA1c level, since the person's last visit.

### SUMMARY

According to an exemplary embodiment of the present disclosure, a method is provided for generating and presenting to a user behavioral insights impacting health outcomes for a person with diabetes, the method comprising: sending an electronic invitation via a network to complete one or more patient reported outcome (PRO) survey instruments to a device associated with the person with diabetes, the PRO survey instruments configured to measure at least one of the person's social, financial, emotional, and psychological state; receiving, at one or more processors via the network, electronic responses to the one or more PRO survey instruments from the device associated with the person; scoring, by the one or more processors, the responses to generate one or more scores associated with the person, wherein each score of the one or more scores measures the extent to which the person is experiencing a different social, financial, emotional, or psychological issue; receiving, by the one or more processors via the network, diabetes treatment information for the person collected over a monitored time period, the diabetes treatment information including at least one of insulin dosage information collected by a connected insulin delivery device and glucose measurement information collected by a connected glucose measurement device; analyzing, by the one or more processors, the diabetes treatment information to derive one or more adverse health outcomes experienced by the person during the monitored time period; automatically generating, by the one or more processors, one or more behavioral insights, wherein each behavioral insight comprises a correlation between one of the derived adverse health outcomes with one or more of the generated scores associated with the person; and generating an indication of the one or more behavioral insights, the generated indication adapted to be presented to the user.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 depicts a system for generating and presenting to a user behavioral insights impacting health outcomes for a person with diabetes, according to some embodiments.

FIG. 2 depicts an exemplary process executed by the system of FIG. 1 for generating and presenting behavioral insights that may impact health outcomes for the person with diabetes, according to some embodiments.

FIGS. 3A and 3B provide a list of published and validated Patient Reported Outcomes (PRO) survey instruments that may be sent to the person with diabetes, according to some embodiments.

FIG. 4A is a table that depicts exemplary social, financial, emotional, and/or psychological issues that belong to a first category of issues pertaining to diabetes management, according to some embodiments.

FIGS. 4B, 4C, 4D, 4E, 4F, and 4G depict exemplary questions that may be posed by one or more PRO survey instruments for assessing issues that belong to the first category of issues, according to some embodiments.

FIG. 5A is a table that depicts exemplary social, financial, emotional, and/or psychological issues that belong to a second category of issues pertaining to diabetes distress, according to some embodiments.

FIG. 5B depicts an exemplary question that may be posed by one or more PRO survey instruments for assessing issues that belong to the second category of issues, according to some embodiments.

FIG. 6 is a table that depicts exemplary social, financial, emotional, and/or psychological issues that belong to a third category of issues pertaining to environmental barriers, according to some embodiments.

FIG. 7 is a table that depicts exemplary social, financial, emotional, and/or psychological issues that belong to a fourth category of issues pertaining to the person's personality or personal style, according to some embodiments.

FIG. 8A is a table that depicts exemplary social, financial, emotional, and/or psychological issues that belong to a fifth category of issues pertaining to the person's mental health, according to some embodiments.

FIG. 8B depicts exemplary questions that may be posed by one or more PRO survey instruments for assessing issues that belong to the fifth category of issues, according to some embodiments.

FIG. 9A is a table that lists exemplary adverse health outcomes and their associated definitions related to the person's glucose levels, according to some embodiments.

FIG. 9B is a table that lists exemplary adverse health outcomes and their associated definitions related to the person's insulin dosing, according to some embodiments.

FIG. 10 is a table that illustrates exemplary logic executed by the system of FIG. 1 for generating behavioral insights, according to some embodiments.

FIG. 11 is a screenshot of an exemplary user-interface for reviewing diabetes-related information for the person with diabetes, according to some embodiments.

FIG. 12 is a screenshot of an exemplary sub-panel in the user-interface for displaying adverse health outcomes detected in the person's diabetes treatment information, according to some embodiments.

FIG. 13 is a screenshot of an exemplary sub-panel in the user-interface for displaying social, financial, emotional, and/or psychological issues that were surfaced by the person's PRO survey instrument responses, and which may be correlated with at least one of the adverse health outcomes depicted in FIG. 12, according to some embodiments.

FIG. 14 is a screenshot that displays the person's scores associated with different social, financial, emotional, and/or psychological issues, according to some embodiments.

FIG. 15 is a block diagram that illustrates the logical components of a server for implementing the process described in FIG. 2.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

Connected glucose monitoring devices and/or connected insulin delivery devices provide persons with diabetes and HCPs with a wealth of data regarding a person's diabetes and treatment. For example, such connected devices may provide more granular and/or accurate diabetes treatment information regarding the person's glucose levels over a monitored time period (e.g., days, weeks, or months) and/or the times and amounts of insulin administered to the person. This wealth of data provides HCPs the opportunity to provide better feedback to patients and better understand where their patients may be experiencing adverse health outcomes. For example, this data may alert HCPs when their patients are experiencing episodes of hypoglycemia, hyperglycemia, or are not meeting their time-in-range or glucose variation goals.

However, diabetes treatment information alone may not provide a complete picture into the factors that are causing and/or exacerbating these observed adverse health outcomes. Many of the adverse health outcomes in persons with diabetes may be improved or mitigated by changing the person's behavior, such as by improving their insulin bolusing or eating practices. Unfortunately, changing the person's behavior may be challenging, as diabetes is a chronic disease that imposes a heavy burden on patients to constantly manage their glucose levels, eating patterns, and insulin doses, among other factors. There may be many complex social, financial, emotional, and/or psychological issues present in the person's life that may be preventing the person from changing her behavior. Diabetes treatment information that comprises glucose and/or insulin dosage data alone does not provide HCPs any insight regarding these issues, thus preventing HCPs from effectively counseling their patients to improve their health outcomes.

For example, an HCP addressing adverse health outcomes associated with the behavior or adverse health outcome of frequent missed insulin boluses (e.g., where the person with diabetes does not take insulin to cover a meal or to correct existing hyperglycemia) may benefit from insight into the root social, financial, emotional, and/or psychological issues that are driving this behavior or outcome. The same behavior or adverse health outcome (missed boluses) may be driven by different issues in different persons with diabetes. Some of the different relevant issues may include (1) a fear or lack of confidence in managing hypoglycemic episodes, (2) a desire to avoid social stigma, such as a desire to avoid feeling abnormal in social situations, to not interrupt the spontaneity of situations, or to avoid feelings of embarrassment, (3) a desire to reduce or omit boluses in order to avoid weight gain, (4) diabetes exhaustion, such as feelings of tiredness from never having a break from managing one's diabetes, or (5) feelings that managing one's diabetes are not worth the effort, such as frustration that bolusing efforts do not produce desired results, a belief that elevated glucose levels are not dangerous, or feeling that one is too busy to manage boluses. A person with diabetes may struggle with none, some, or all of the foregoing issues.

Different treatments and/or counseling may be appropriate for the same behavior or outcome (e.g., frequent missed insulin boluses) depending on the root social, financial, emotional, and/or psychological issues that are driving this behavior. For example, if the person feels that managing one's diabetes is not worthwhile because elevated glucose levels are not dangerous, the correct approach for the HCP to take may be to better educate the person regarding the short- and long-term consequences of elevated glucose levels. That same approach, however, may not be appropriate for persons that are suffering from diabetes exhaustion, as that may simply contribute to the person's guilt and frustration. Rather, providing tools or education regarding tools and processes to relieve the complexity and burden of managing diabetes may be a more effective means of addressing diabetes exhaustion. Such tools and processes could include, for example, bolus advisors or calculators, reminders, or other personalized solutions that decrease the burden of managing diabetes. As another example, if the person is intentionally missing boluses because of a fear or lack of confidence in managing hypoglycemic episodes, the correct approach may be to better educate or train the person regarding how to catch and treat hypoglycemic episodes, or provide better tools and processes for monitoring for hypoglycemic episodes (e.g., prescribing use of a continuous glucose monitor). On the other hand, this approach would be ineffective if the person is missing boluses out of a desire to avoid social stigma. The better approach in these cases may be to provide counseling that normalizes the feelings of shame or embarrassment the person has regarding his or her diabetes.

Although important for providing effective counseling and treatment for mitigating adverse health outcomes, such insights regarding root social, financial, emotional, and/or psychological issues cannot be discerned solely from glucose measurements and insulin dosage data. Therefore, a need exists for methods and systems to obtain these insights into root social, financial, emotional, and/or psychological issues that may be driving or exacerbating adverse health outcomes. Furthermore, a need exists for methods and systems to correlate these insights with adverse health outcomes detected in the person's diabetes treatment information. Correlating these insights enables HCPs to have richer, more effective conversations with their patients that are more likely to change their patients' behavior.

The terms “logic,” “control logic,” “application,” “process,” “method,” “algorithm,” and “instructions” as used herein may include software and/or firmware executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion and would remain in accordance with the embodiments herein disclosed.

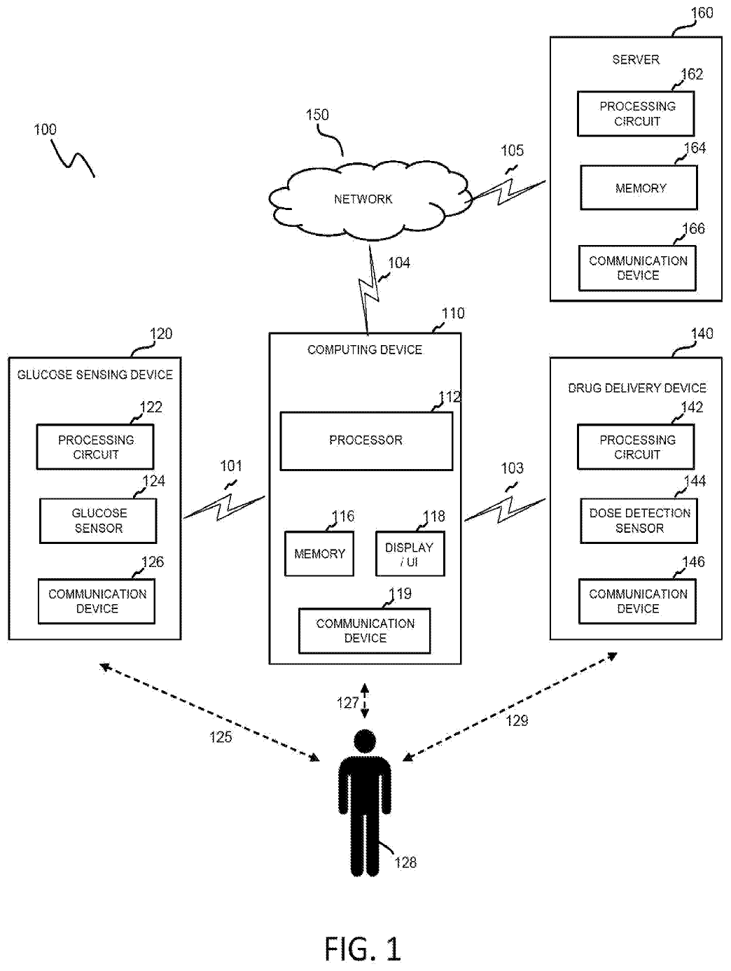

FIG. 1 depicts a system 100 for generating and presenting to a user behavioral insights impacting health outcomes for a person with diabetes, according to some embodiments. System 100 includes a computing device 110 in wireless communication with a connected glucose sensing device 120 and/or a connected drug delivery device 140. Computing device 110 may also be in communication with a server 160 via a network 150.

Computing device 110 illustratively includes a mobile device, such as a smartphone. Alternatively, any suitable computing device may be used, including but not limited to a laptop, desktop, tablet, or server computer, for example. Computing device 110 includes processor 112, memory 116, display/user-interface (UI) 118, and communication device 119.

Processor 112 includes at least one processor that executes software and/or firmware stored in memory 116 of computing device 110. The software/firmware code contains instructions that, when executed by processor 112, causes processor 112 to perform the functions described herein. Such instructions illustratively include collecting diabetes treatment information from one or both of glucose sensing device 120 and drug-delivery device 140 and transmitting such diabetes treatment information to server 160 via network 150. Such instructions may also illustratively include providing a user-interface that allows a user of computing device 110 to receive and respond to one or more patient reported outcome (PRO) survey instruments, as discussed in more detail below. Memory 116 is any suitable computer readable medium that is accessible by processor 112. Memory 116 may be a single storage device or multiple storage devices, may be located internally or externally to processor 112, and may include both volatile and non-volatile media. Exemplary memory 116 includes random-access memory (RAM), read-only memory (ROM), electrically erasable programmable ROM (EEPROM), flash memory, a magnetic storage device, optical disk storage, or any other suitable medium which is configured to store data, and which is accessible by processor 112.

Computing device 110 includes a display/user interface 118 in communication with processor 112 and operative to provide user input data to the system and to receive and display data, information, and prompts generated by the system. User interface 118 includes at least one input device for receiving user input and providing the user input to the system. In the illustrated embodiment, user interface 118 is a graphical user interface (GUI) including a touchscreen display operative to display data and receive user inputs. The touchscreen display allows the user to interact with presented information, menus, buttons, and other data to receive information from the system and to provide user input into the system. Alternatively, a keyboard, keypad, microphone, mouse pointer, or other suitable user input device may be provided.

Computing device 110 further includes communication device 119 that allows computing device 110 to establish wired and/or wireless communication links with other devices. Communication device 119 may comprise one or more wireless antennas and/or signal processing circuits for sending and receiving wireless communications, and/or one or more ports for receiving physical wires for sending and receiving data. Using communication device 119, computing device 110 may establish one or more short-range communication links, including one or more of communication link 101 with glucose sensing device 120, and communication link 103 with drug delivery device 140. Such short-range communication links may utilize any known wired or wireless communication technology or protocol, including without limitation radio frequency communications (e.g., Wi-Fi, Bluetooth, Bluetooth Low Energy (BLE), Near Field Communications (NFC), RFID, and the like), infrared transmissions, microwave transmissions, and lightwave transmissions. Such short-range communication links may be either uni-directional links (e.g., data flows solely from glucose sensor 120, and/or device 140 to computing device 110), or bi-directional links (e.g., data flows both ways). Communication device 119 may also allow computing device 110 to establish a long-range communication link with a server 160 via a network 150, and communication links 104 and 105. The server 160 may be located remote from computing device 110, e.g., in another building, in another city, or even in another country or continent. Network 150 may comprise any cellular or data network adapted to relay information from computing device 110 to and/or from server 160, potentially via one or more intermediate nodes or switches. Examples of suitable networks 150 include a cellular network, a metropolitan area network (MAN), a wide area network (WAN), and the Internet.

Connected glucose sensor 120 illustratively includes any sensor adapted to measure a glucose level of a person with diabetes, such as a blood glucose monitor (BGM), a continuous glucose monitor (CGM), and/or a flash glucose monitor (FGM). Glucose sensor 120 includes a processing circuit 122, a glucose sensor 124, and communication device 126. Processing circuit 122 may include any processing circuit that receives and processes data signals, and which outputs results in the form of one or more electrical signals as a result. Processing circuit 122 may include a processor (similar to processor 112), an Application Specific Integrated Circuit (ASIC), field-programmable gate arrays (FPGAs), digital signal processors (DSPs), hardwired logic, or combinations thereof. Glucose sensor 124 comprises any sensor capable of extracting and/or analyzing analyte (e.g., blood or interstitial fluid) from the body of the person with diabetes to measure and/or record the person's glucose levels. Communication device 126 allows glucose sensor 120 to communicate with computing device 110 via communication link 101, and to relay the measured glucose levels to computing device 110.

Drug delivery device 140 illustratively includes any device configured to deliver a dose of insulin to a person with diabetes, to measure and/or record the time and amount of dose delivered, and to communicate this information to computing device 110. The term “insulin” refers to one or more therapeutic agents including insulins, insulin analogs such as insulin lispro or insulin glargine, and insulin derivatives. Such a device may be operated by a patient, caregiver or healthcare professional to deliver insulin to a person. The insulin delivered by device 140 may be formulated with one or more excipients. Drug delivery device 140 may be configured as a re-usable device that may be re-filled with insulin once its store of insulin is exhausted or may be configured as a disposable device that is designed to be discarded and replaced once its store of insulin is exhausted. Drug delivery device 140 includes processing circuit 142, dose detection sensor 144, and communication device 146. Processing circuit 142 may include any of the possible types of processing circuits previously described. Dose detection sensor 144 may include any suitable sensor for detecting and/or recording the time and amount of dose delivered. Communication device 146 allows drug delivery device 140 to communicate with computing device 110 via communication link 103.

Server 160 illustratively includes any computing device configured to receive information regarding a person with diabetes from computing device 110 via network 150, process said information, and optionally, to send responses, notifications, or instructions to computing device 110 in response to said information. Server 160 may also be configured to send reports, data, and/or notifications to a HCP (not shown), either through a user-interface local to server 160, or via a web- or remote portal viewable through a remote device associated with the HCP (also not shown). Server 160 includes processing circuit 162, memory 164, and communication device 166. Processing circuit 162 may include any of the possible types of processing circuits previously described, and may also include multiple processing circuits (e.g., multiple processors). Processing circuit 162 may execute software and/or firmware stored in memory 164 of server 160. The software/firmware code contains instructions that, when executed by processing circuit 162, cause processing circuit 162 to perform the functions described herein. Memory 164 may also be configured to store information regarding one or more persons with diabetes, such as biographical information and/or medical information (e.g., insulin dosing records, medical history, and the like). Information received from or sent to computing device 110 may also be stored in memory 164. Memory 164 may include any of the possible types of memory previously described. Communication device 166 allows server 160 to communicate with computing device 110 via communication link 105, network 150, and communication link 104.

As depicted by double-ended-and-dashed association arrows 125, 127, and 129 respectively, connected glucose sensing device 120, computing device 110, and connected drug-delivery device 140 are each associated—e.g., by ownership, possession, and/or in one or more other ways—with a person with diabetes 128.

In some embodiments, system 100 may be modified by omitting one or both of glucose sensing device 120 and drug delivery device 140. For example, instead of using a connected glucose sensing device 120 as shown, a user of system 100 may instead measure or estimate his/her own glucose levels using other methods (e.g., using a non-connected glucose sensing device, such as a BGM), and then manually input the measured glucose level and the time of measurement into computing device 110. As another example, instead of using a connected drug delivery device 140 as shown, a user of system 100 may instead manually inject him or herself using a non-connected delivery device (e.g., a syringe), and then manually input the time and amount of insulin doses taken.

In other embodiments, system 100 may be modified by adding components. For example, server 160 may be configured as a plurality of networked servers 160 that cooperate to process information. Such a configuration of networked servers may be referred to as a “cloud” of servers that perform the functions described herein. The server(s) 160 may communicate with multiple computing devices 110 via network 150, and each computing device 110 may in turn be optionally connected with one or more glucose sensing devices 120 and one or more drug delivery devices 140.

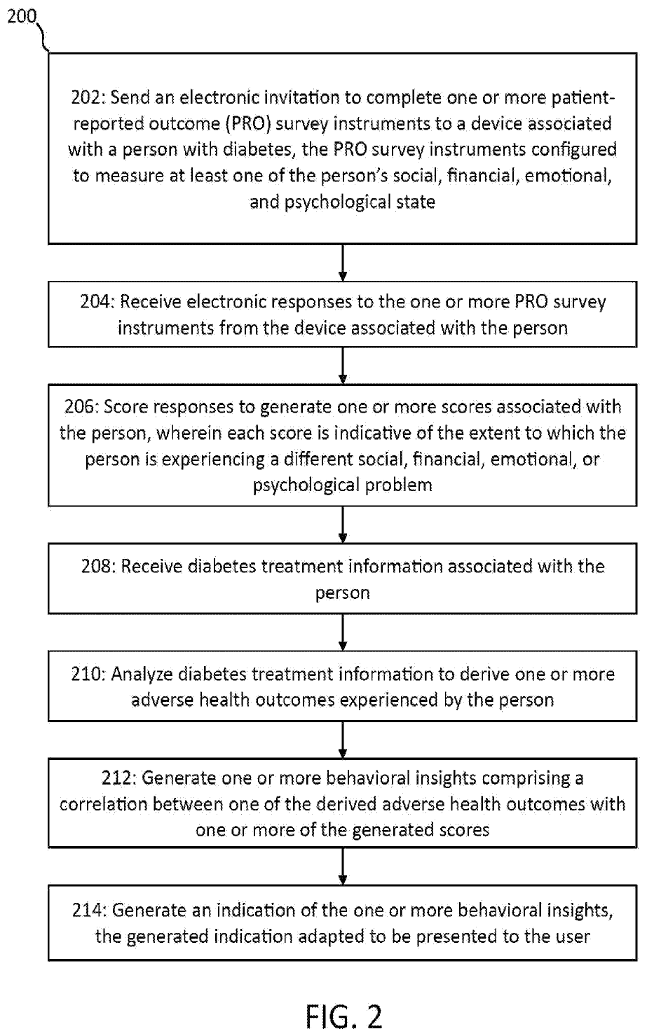

FIG. 2 depicts an exemplary process 200 for generating and presenting behavioral insights that may impact health outcomes for a person with diabetes, according to some embodiments. Process 200 may be implemented on server 160, with input from other devices depicted in FIG. 1.

Process 200 begins at step 202 where server 160 sends an electronic invitation to complete one or more Patient Reported Outcome (PRO) survey instruments to a device associated with a person with diabetes (e.g., to computing device 110 associated with person 128). A PRO survey instrument may comprise an electronic questionnaire inquiring about different aspects of a person's social, financial, emotional, and/or psychological state (e.g., the person's history, self-reported tendencies, proclivity to struggle with a certain type of issue, etc.). These questions may request the person to rate their response on a numerical scale (e.g., select a number between 1 and 6 depending on how much they struggle with a particular issue), or select a statement out of a short list of presented statements that best applies to her (e.g., “I always have a problem with X”, “I sometimes have a problem with X”, or “I never have a problem with X.”). The PRO survey instrument may also comprise information and/or documentation that supports its use, such as instructions to the person taking the survey as well as the person interpreting the survey. PRO survey instruments may be used to capture patient-reported data used to measure treatment benefit or risk. In particular, the PRO survey instruments may measure aspects of the person's social, financial, emotional, and/or psychological state that may impact treatment and/or health outcomes related to the person's diabetes.

The invitation to complete the one or more PRO survey instruments may take different forms. For example, the invitation may be sent to the person via a SMS text message, an email, or an instant message through a suitable instant messaging service (e.g., iMessage, Skype, FaceTime, WhatsApp, WeChat, etc.) that includes a hyperlink. When the hyperlink is activated by the person on the person's computing device, the person's computing device may be prompted to open a webpage or portal at which the person may access the one or more PRO survey instruments and complete them. Alternatively, or in addition, the invitation may be sent directly to a mobile application installed on the person's computing device. The mobile application may then prompt the person through a user notification (e.g., through an audible chime, a haptic tap, or a flashing light) to access and complete the one or more PRO survey instruments.

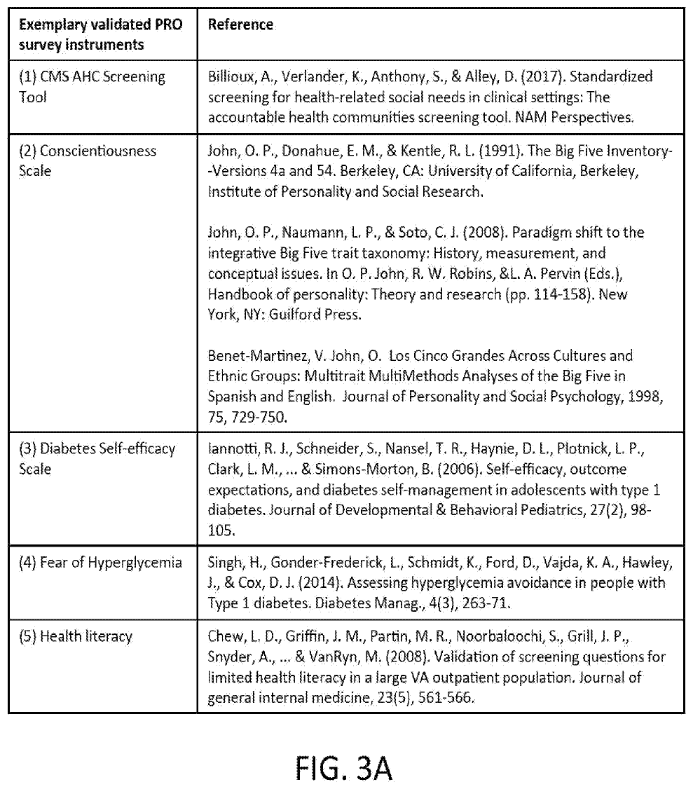

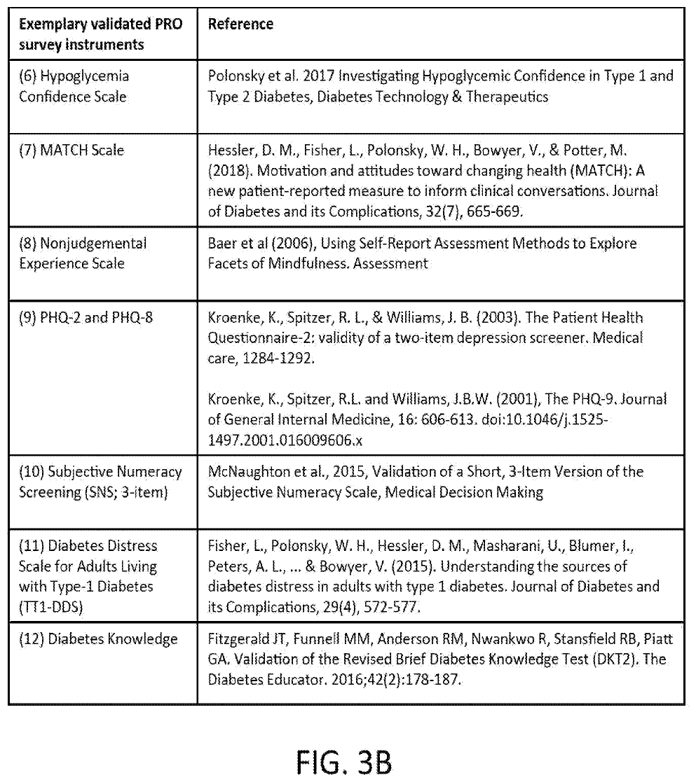

FIGS. 3A and 3B provide a list of exemplary published and validated PRO survey instruments that may be sent to the person at step 202. Each of the listed references, as well as the PRO survey instruments described therein, are incorporated by reference herein in their entirety for all purposes. As used herein, the term “validated PRO survey instrument” may refer to a PRO survey instrument that has been studied by members of the scientific and/or academic community, and evidence exists to prove that such a PRO survey instrument validly measures what it says it does, and that its results are reliable. Some of these PRO survey instruments may be specific to diabetes, such as instruments (3), (4), (6), (7), and (11). Other PRO survey instruments may assess the person's general social, financial, emotional, and/or psychological state, without specific reference to diabetes. The electronic invitation may ask the person to complete all or a part of a PRO survey instrument, e.g., in a PRO survey instrument comprising multiple questions, the person may be requested to provide responses for only a subset of the included questions. Other PRO survey instruments that have not been previously published or validated may also be used at step 202.

At step 204, server 160 receives electronic responses to the one or more PRO survey instruments from the device associated with the person. The electronic responses may comprise answers to questions posed in the questionnaire portion of the PRO survey instruments. These electronic responses may be saved in memory communicably coupled to server 160, e.g., memory 164.

At step 206, server 160 scores the received responses to generate one or more scores associated with the person, wherein each score is indicative of the extent to which the person is experiencing a different social, financial, emotional, or psychological problem. As used herein, a “score” may comprise a number within a specified range of numbers (e.g., a number between 1 and 5), a letter grade within a specified range of letter grades (e.g., a letter between A and F), a selection of one statement from within a specified set of statements (e.g., a selection between the statements “Always a problem”, “Often a problem”, “Rarely a problem”, and “Never a problem”), a binary indicator (e.g., “Yes/No”, “True/False”, “Present/Not Present”), and the like. In some embodiments, one score may be generated from responses to one or more questions from a single PRO. In other embodiments, one score may be generated from the received responses for multiple PROs. In other embodiments, multiple scores may be generated from a single PRO.

The scores may be generated in multiple ways. For example, a generated score may simply equal the person's numerical rating response to a particular question. For questions that do not elicit a numerical response, the person's response may be converted into a numerical rating. For instance, if a particular question asks the person to select between the statements “Always a problem”, “Often a problem”, “Rarely a problem”, and “Never a problem”, a score may be generated by assigning the numerical rating (4) to the first statement, the rating (3) to the second statement, the rating (2) to the third statement, and the rating (1) to the last statement. In some situations, a score may be generated by calculating the mean or median average of the person's numerical response to multiple questions. A score may also be generated by taking the maximum or minimum numerical response to a set of questions. Where appropriate, generating a score may involve normalizing the person's numerical response from one scale to another scale (e.g., from a 6-point scale to a 10-point scale), or inverting the person's numerical score (e.g., instead of 1 being “Not a Problem” and 6 being “A Very Serious Problem”, the score may be inverted such that 1 means “A Very Serious Problem” and 6 means “Not a Problem.”). A score may also be generated by counting the number of statements of a certain type (e.g., the number of affirmative or negative responses to a set of questions), or by adding the person's responses over multiple questions. Scores may also be generated by performing other mathematical operations on the person's numerical response, such as addition, subtraction, multiplication, and/or division. Any of the foregoing operations may be used in any combination, and in any order, to generate a score.

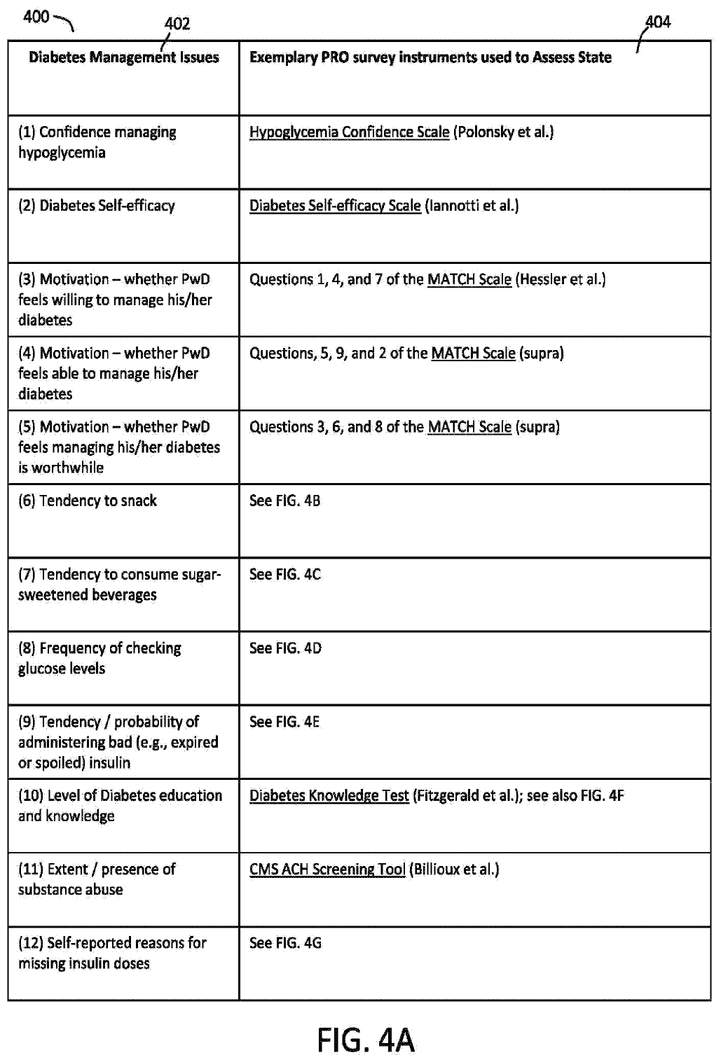

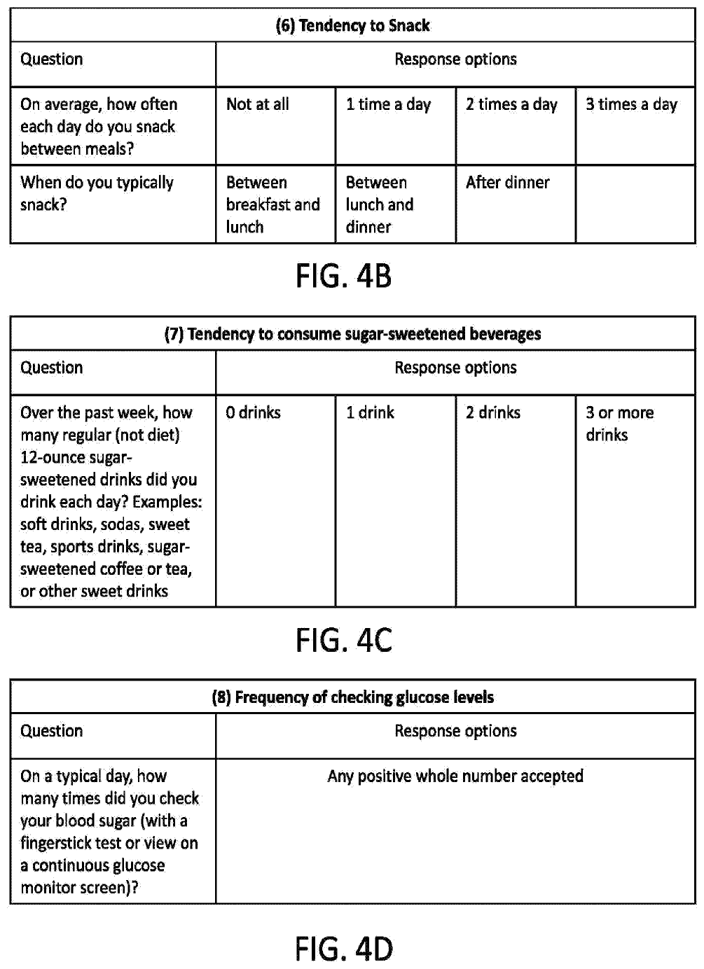

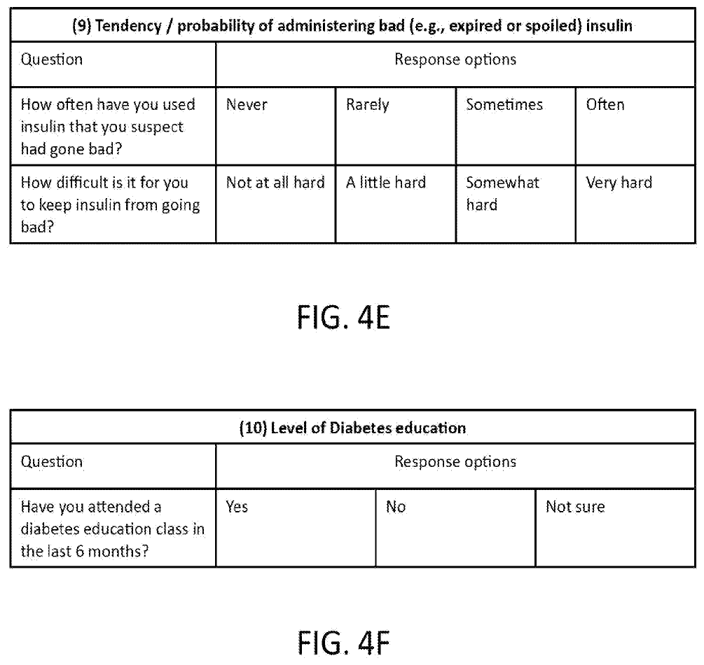

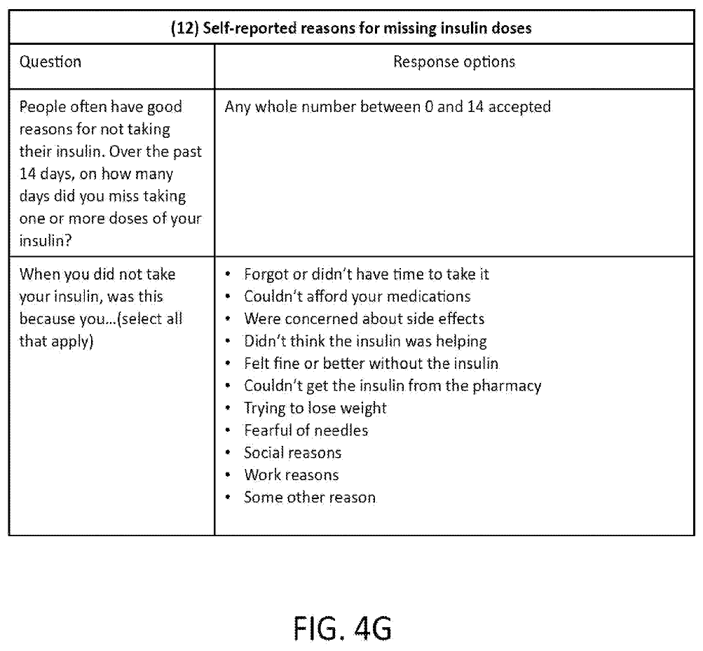

FIG. 4A is a table 400 that depicts twelve exemplary social, financial, emotional, and/or psychological issues 402 that belong to a first category of issues pertaining to diabetes management, e.g., how effectively the person manages the day-to-day tasks associated with his/her diabetes. By scoring the person's responses to the PRO survey instruments, process 200 may assign a different score to each of these twelve issues. Each score indicates the extent to which the person experiences problems associated with that corresponding issue.

As an illustrative example, issue (1) depicted in FIG. 4A pertains to the person's confidence in managing hypoglycemia. The extent to which the person experiences problems with this issue may be evaluated by scoring the person's responses to the Hypoglycemia Confidence Scale (Polonsky et al.). This scale comprises five questions that ask the person to indicate how confident she is that she can stay safe from serious problems with hypoglycemia when (1) exercising, (2) sleeping, (3) driving, (4) in social situations, and (5) alone. The person indicates her level of confidence by selecting one of four options for each question: “Not confident at all”, “A little confident”, “Moderately confident”, and “Very confident.” When scoring the person's response, process 200 can assign a numerical rating of (1) to the statement “Not confident at all”, a rating of (2) to the statement “A little confident”, a rating of (3) to the statement “Moderately confident”, and a rating of (4) to the statement “Very confident.” These numerical ratings can then be averaged across the five questions in the Hypoglycemia Confidence Scale to generate a single, aggregate numerical score (from 1 to 4) that indicates the person's confidence in avoiding serious problems with hypoglycemia.

A similar process may be used to generate scores for each of the remaining issues (2) through (12) in FIG. 4A. For example, issue (2) of FIG. 4A, pertaining to the person's level of diabetes self-efficacy, may be evaluated by scoring the person's responses to the Diabetes Self-efficacy Scale (Iannotti et al.). Issue (3) of FIG. 4A, pertaining to the person's level of motivation to manage her diabetes, may be evaluated by scoring the person's responses to questions 1, 4, and 7 of the MATCH scale (Hessler et al.). In each case, a single aggregate numerical score for each issue may be calculated using any of the aforementioned techniques, e.g., by assigning numerical ratings to selected statements and aggregating these numerical ratings into a single representative number (e.g., by calculating an average, a sum, a product, etc.)

In this embodiment, issues (6), (7), (8), (9), (10), and (12) may not evaluated using a previously published and validated PRO survey instrument. Rather, issue (6) may be evaluated using the questions depicted in FIG. 4B, issue (7) may be evaluated using the question depicted in FIG. 4C, issue (8) may be evaluated using the question depicted in FIG. 4D, issue (9) may be evaluated using the questions depicted in FIG. 4E, issue (10) may be evaluated either the Diabetes Knowledge Test (Fitzgerald et al.) or using the questions depicted in 4F, and issue (12) may be evaluated using the questions depicted in FIG. 4G.

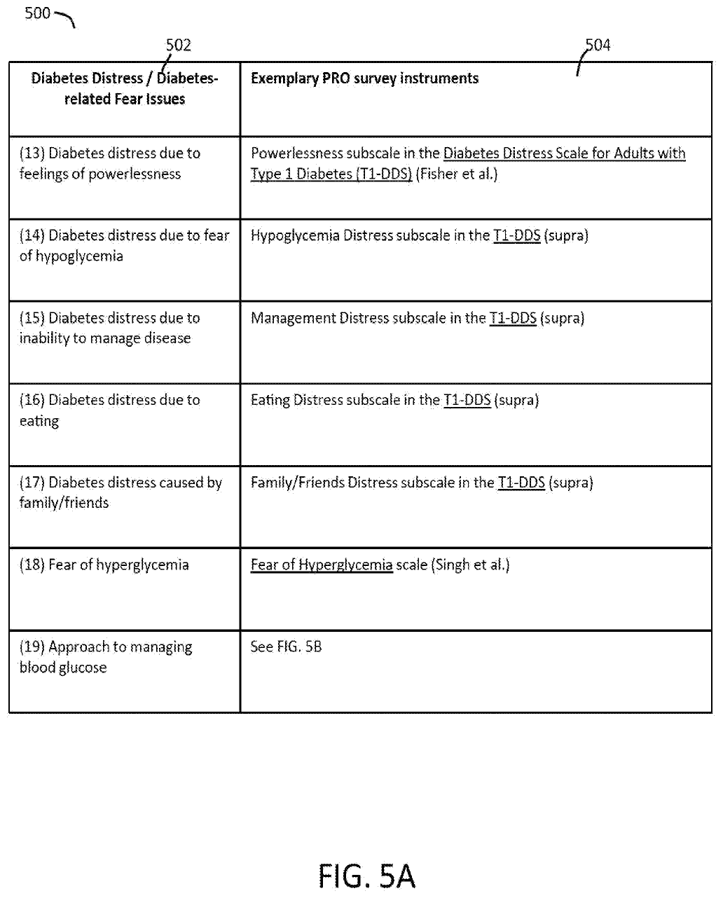

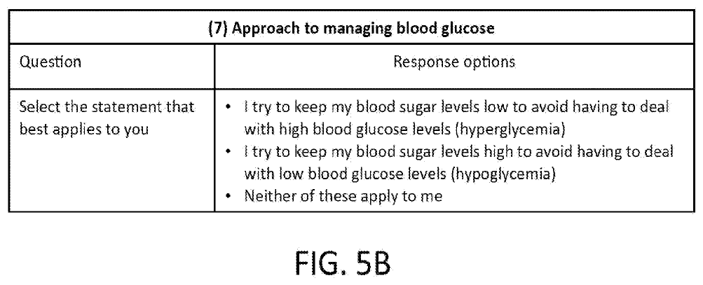

FIG. 5A is a table 500 that depicts seven exemplary social, financial, emotional, and/or psychological issues 502 (i.e., issues (13) through (19)) that belong to a second category of issues pertaining to diabetes-related distress or fear, e.g., common areas of psychological “stress” that persons with diabetes encounter. Again, by scoring the person's responses to the PRO survey instruments, process 200 may assign a different score to each of these seven issues. Each score indicates the extent to which the person experiences problems associated with that corresponding issue.

As another illustrative example, issue (13) in FIG. 5A pertains to whether the person experiences diabetes distress due to feelings of powerlessness. The extent to which the person experiences problems with this issue may be evaluated by scoring the person's responses to the Powerlessness sub scale of the Diabetes Distress Scale for Adults with Type 1 Diabetes (T1-DDS) (Fisher et al.). The T1-DDS comprises multiple sub-scales, each associated with a different type of distress, e.g., “Powerlessness”, “Hypoglycemia Distress”, “Management Distress”, “Eating Distress”, and the like. The Powerlessness subscale asks the person to indicate the extent to which each of the following five issues is a problem in their life: (1) “feeling that I've got to be perfect with my diabetes management”, (2) “feeling that no matter how hard I try with my diabetes, it will never be good enough”, (3) “feeling discouraged when I see high blood glucose numbers that I can't explain”, (4) “feeling that there is too much diabetes equipment and stuff I must always have with me”, and (5) “feeling worried that I will develop serious long-term complications, no matter how hard I try.” The person can respond to each question by selecting between one of six categories: “Not a problem” (assigned a numerical rating of 1), “A slight problem” (numerical rating of 2), “A moderate problem” (numerical rating of 3), “A somewhat serious problem” (numerical rating of 4), “A serious problem” (numerical rating of 5), and “A very serious problem” (numerical rating of 6). As discussed previously, the person's numerical rating for each question may then be aggregated into a single representative score (e.g., by calculating a mean average) that represents the extent to which the person struggles with feelings of powerlessness in managing her diabetes.

A similar process may be used to generate scores for each of the remaining issues (14) through (19) in FIG. 5A. For example, the second issue, pertaining to the person's level of diabetes distress due to fear of hypoglycemia, may be evaluated by scoring the person's responses to the Hypoglycemia Distress subscale in the T1-DDS. In each case, a single aggregate numerical score for each issue may be calculated using any of the aforementioned techniques.

In this embodiment, issue (19) (pertaining to the person's approach in managing their blood glucose) is not evaluated using a previously published PRO survey instrument. Rather, issue (19) may be evaluated using the questions depicted in FIG. 5B.

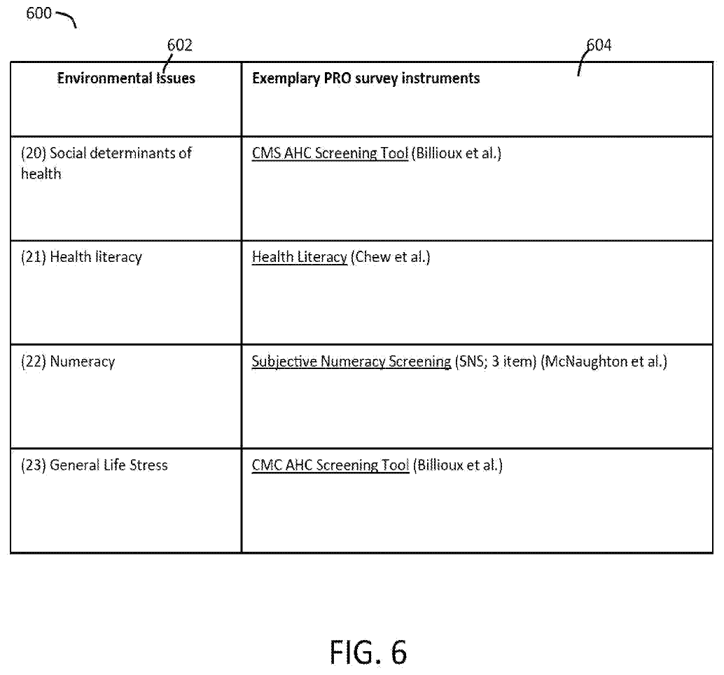

FIG. 6 is a table 600 that depicts four exemplary social, financial, emotional, and/or psychological issues 602 (i.e., issues (20) through (23)) that belong to a third category of issues pertaining to environmental barriers. As before, process 200 may assign a different score to each of these four issues. Each score indicates the extent to which the person experiences problems associated with that corresponding issue. Issue (20), pertaining to social determinants of health (e.g., the environmental and structural elements of our lives that impact our health, such as stability/security of the person's housing, food, transportation, utilities, education, employment, neighborhood, community, and the like) may be evaluated by scoring the person's responses to the CMS AHC Screening Tool (Billioux et al.). Issue (23), pertaining to health literacy, may be evaluated by scoring the person's responses to the Health Literacy PRO survey instrument (Chew et al.).

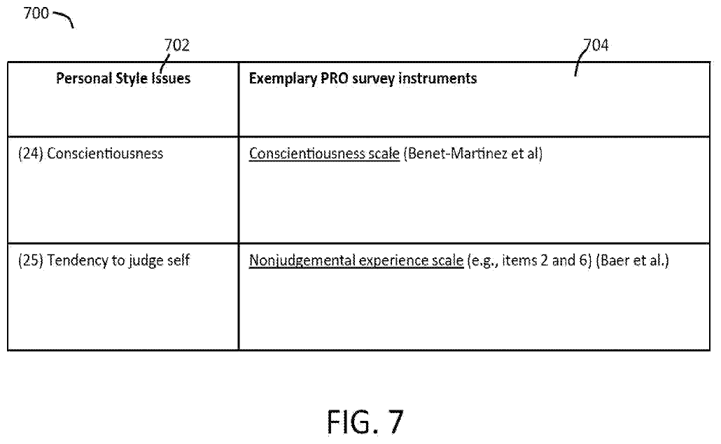

FIG. 7 is a table 700 that depicts two exemplary social, financial, emotional, and/or psychological issues 702 (i.e., issues (24) and (25)) that belong to a fourth category of issues pertaining to the person's personality or personal style. As before, process 200 may assign a different score to each of these two issues. Each score indicates the extent to which the person experiences problems associated with that corresponding issue. Issue (24), pertaining to the person's level of conscientiousness (e.g., whether the person does a thorough job, makes plans and follows through with them, and/or perseveres until a task is finished) may be evaluated by scoring the person's responses to the Conscientiousness scale (Donahue et al.; Naumann et al.; Benet-Martinez et al.). Issue (25), pertaining to the person's tendency to judge him or herself, may be evaluated by scoring the person's nonjudgmental experience scale (e.g., questions 2 and 6) (Baer et al.).

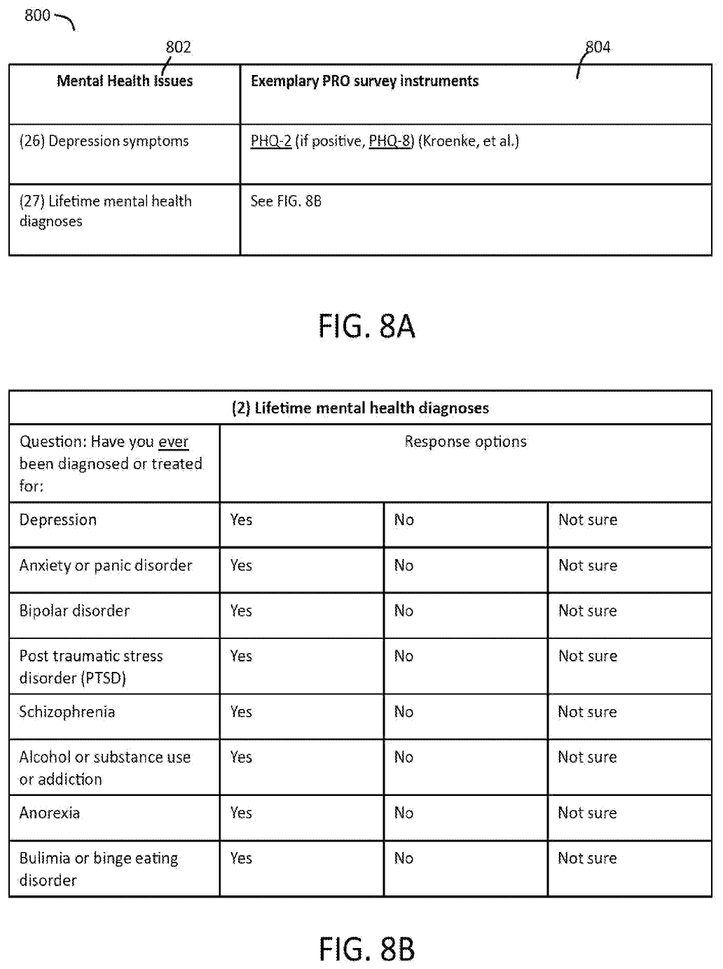

FIG. 8A is a table 800 that depicts two exemplary social, financial, emotional, and/or psychological issues 802 (i.e., issues (26) and (27)) that belong to a fifth category of issues pertaining to the person's mental health. As before, process 200 may assign a different score to each of these 2 issues. Each score indicates the extent to which the person experiences problems associated with that corresponding issue. Issue (26), pertaining to whether the person is experiencing symptoms associated with depression, may be evaluated by scoring the person's responses to the PHQ-2 PRO survey instrument (Kroenke et al. (2003)). If the person's responses indicate the person is experiencing symptoms associated with depression, the person may be further prompted to complete the PHQ-8 PRO survey instrument (Kroenke et al. (2001)). Issue (28), pertaining to whether the person has ever been diagnosed with mental health issues in her lifetime, may be evaluated by scoring the person's responses to the questions presented in FIG. 8B.

Returning to FIG. 2, at step 208, server 160 receives diabetes treatment information associated with the person. The diabetes treatment information may include at least one of insulin dosage information collected by a connected insulin delivery device (e.g., device 140) and glucose measurement information collected by a connected glucose measurement device (e.g., device 120) over a monitored time period (e.g., days, weeks, or months).

At step 210, process 200 analyzes the diabetes treatment information to derive one or more adverse health outcomes experienced by the person. The adverse health outcomes may be derived from the glucose measurement information by calculating the number, frequency, duration, and/or severity of episodes of hypoglycemia (where glucose levels are lower than normal or desirable) and/or episodes of hyperglycemia (where glucose levels are higher than normal or desirable) during the monitored time period, and determining whether such episodes exceed certain predetermined criteria or thresholds. The adverse health outcomes may also be derived by calculating whether the variation of the person's glucose levels over the monitored time period, e.g., by calculating a variance, range, standard of deviation, or a Coefficient of Variance (CV) (e.g., calculated by dividing the standard deviation of the person's glucose levels over the monitored time period by the mean of the person's glucose levels over the monitored time period), exceed certain predetermined criteria or thresholds. The adverse health outcomes may also be derived by determining whether the percentage of time during the monitored time period during which the person's glucose levels were within a desirable range (“time-in-range”), such as 70-180 mg/dL, satisfy one or more predetermined criteria or thresholds. In some embodiments, the adverse health outcomes may be derived from both the glucose measurement information and insulin dosage information. For example, the adverse health outcomes may be derived by comparing the time-of-onset of hyperglycemic or hypoglycemic episodes, or the time-of-onset of rapid upward or downward changes in glucose levels, with the time and/or amount of insulin boluses. This comparison may reveal times when the person may have missed a bolus, administered a late bolus, administered an insufficient bolus (resulting in a prolonged hyperglycemic episode), administered an excessive bolus (resulting in a hypoglycemic episode), and/or “stacked” multiple boluses by administering multiple boluses over too short a time period (again, resulting in a hypoglycemic episode). In yet other embodiments, the adverse health outcomes may be derived by comparing the amount of insulin recommended for a bolus by a bolus calculator and the amount of insulin actually administered to the person. This comparison may reveal times when the person administered more or less insulin than recommended by a bolus calculator. By analyzing changes in the person's glucose levels when the person decided to administer more or less insulin than recommended, process 200 may highlight instances where the person's decision led to undesirable changes in the person's glucose levels.

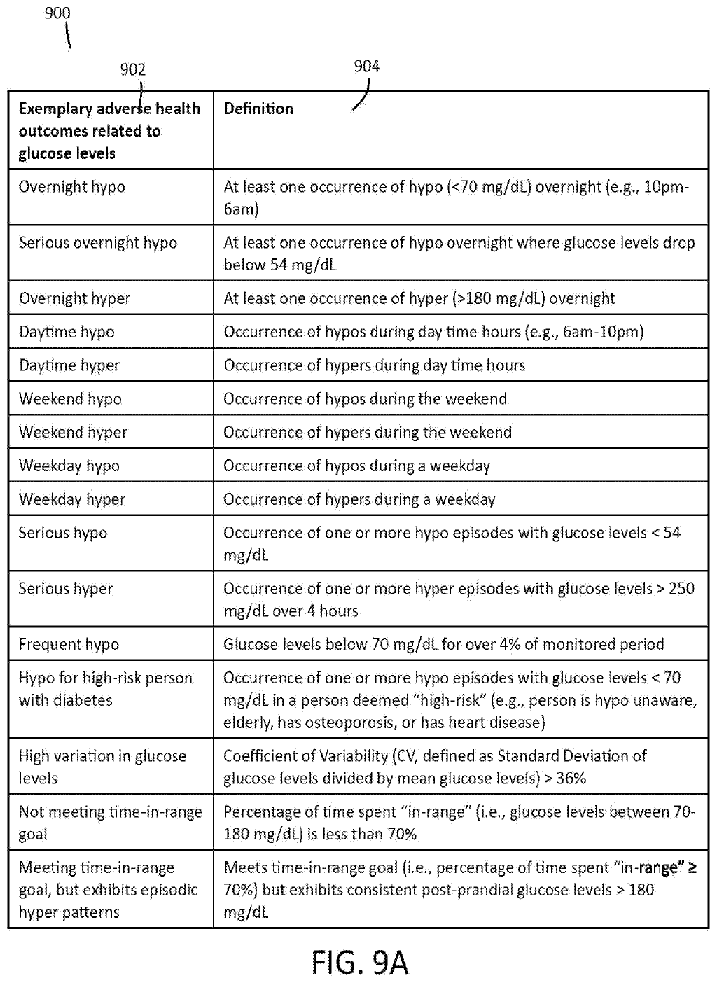

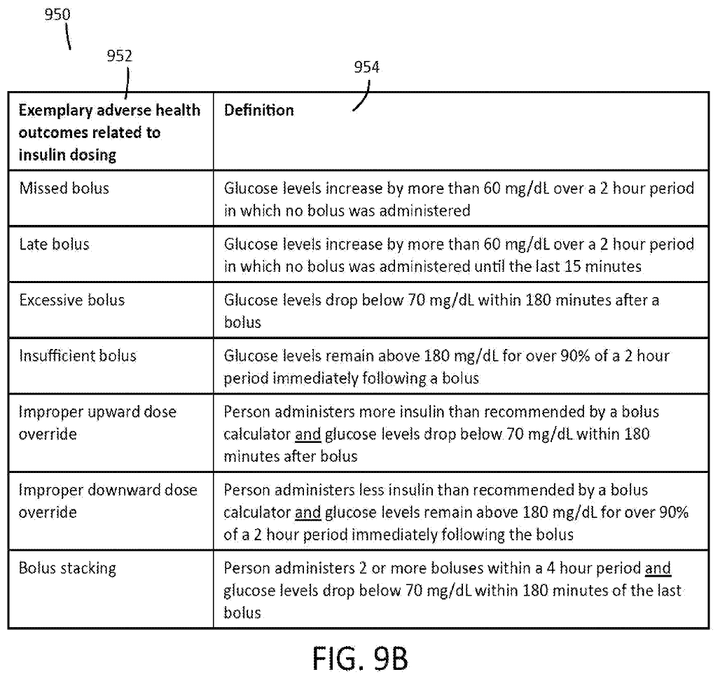

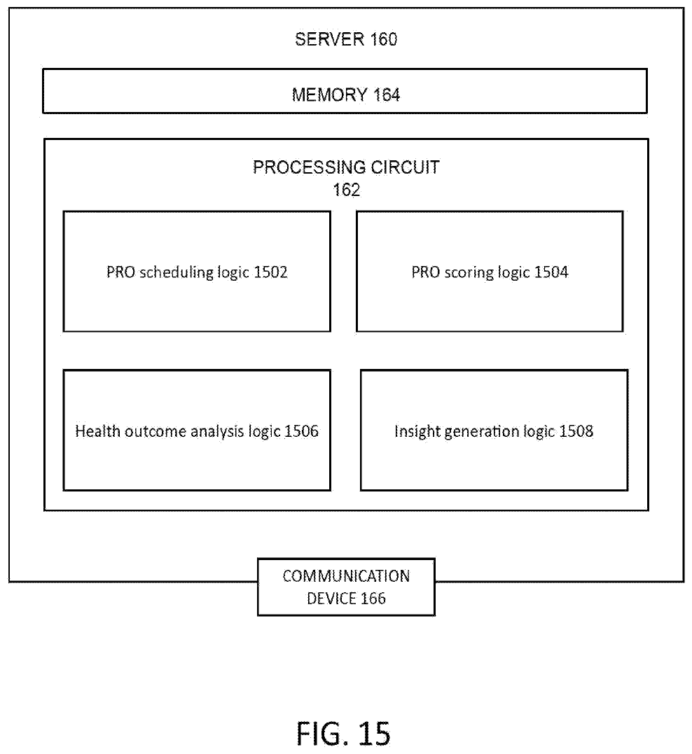

FIGS. 9A and 9B provide exemplary health outcomes that may be derived by health outcome analysis logic 1506 (see FIG. 15) from the person's diabetes treatment information and stored at memory 164 of server 160. FIG. 9A is a table 900 that lists exemplary adverse health outcomes 902 and their associated definitions 904 related to the person's glucose levels, and which may be derived from data collected from a connected glucose measurement device. FIG. 9B is a table 950 that lists exemplary health outcomes 952 and their associated definitions 954 related to the person's insulin dosing, and which may be derived from data collected from both a connected glucose measurement device and a connected drug delivery device. The exemplary health outcomes 902 and 952 may be altered by changing any of the listed numerical thresholds, e.g., with respect to glucose levels, time ranges, percentages, and/or number of required occurrences.

At step 212, server 160 generates one or more behavioral insights comprising a correlation between one of the derived adverse health outcomes with one or more of the generated scores. This may comprise determining, for each of the adverse health outcomes derived from the person's diabetes treatment information, a set of social, financial, emotional, and/or psychological issues that, if present in that person's life, may be relevant to that adverse health outcome. A social, financial, emotional, and/or psychological issue may be relevant to an adverse health outcome if it is expected to be correlated with, cause, and/or exacerbate that adverse health outcome. For example, the issues of (1) low confidence in managing hypoglycemia and/or (14) diabetes distress due to fear of hypoglycemia may be relevant to the adverse health outcome of frequent hyperglycemia. This is because the person may be purposely under-dosing on insulin out of fear that she may inadvertently trigger hypoglycemia, thus causing or exacerbating the observed adverse health outcome of frequent hyperglycemia. In some embodiments, process 200 may determine the set of issues that may be relevant to an adverse health outcome by consulting a lookup table stored in memory.

Once the set of issues that may be relevant to a derived adverse health outcome is determined, process 200 then analyzes the scores generated in step 206 to determine whether the person is in fact experiencing problems with any of these relevant issues. This determination may be done by comparing the person's scores with one or more pre-determined thresholds or criteria. Continuing with the example in the previous paragraph, if the person's score associated with issue (14) (“diabetes distress due to fear of hypoglycemia”) are higher than a certain threshold Y, the person may be considered to be experiencing problems with issue (14). If the person's scores indicate the person is in fact struggling with one or more of the set of relevant issues, process 200 generates a behavioral insight comprising a correlation between the derived adverse health outcome (in this example, “frequent hyperglycemia”) with one or more of the generated scores (in this example, the person's scores associated with issue (14) (“diabetes distress due to fear of hypoglycemia”)). The thresholds used in this determination may be a minimum threshold or a maximum threshold. Alternatively, the thresholds used may be a criteria that the person's score fall within a certain range of values, or outside of a certain range of values.

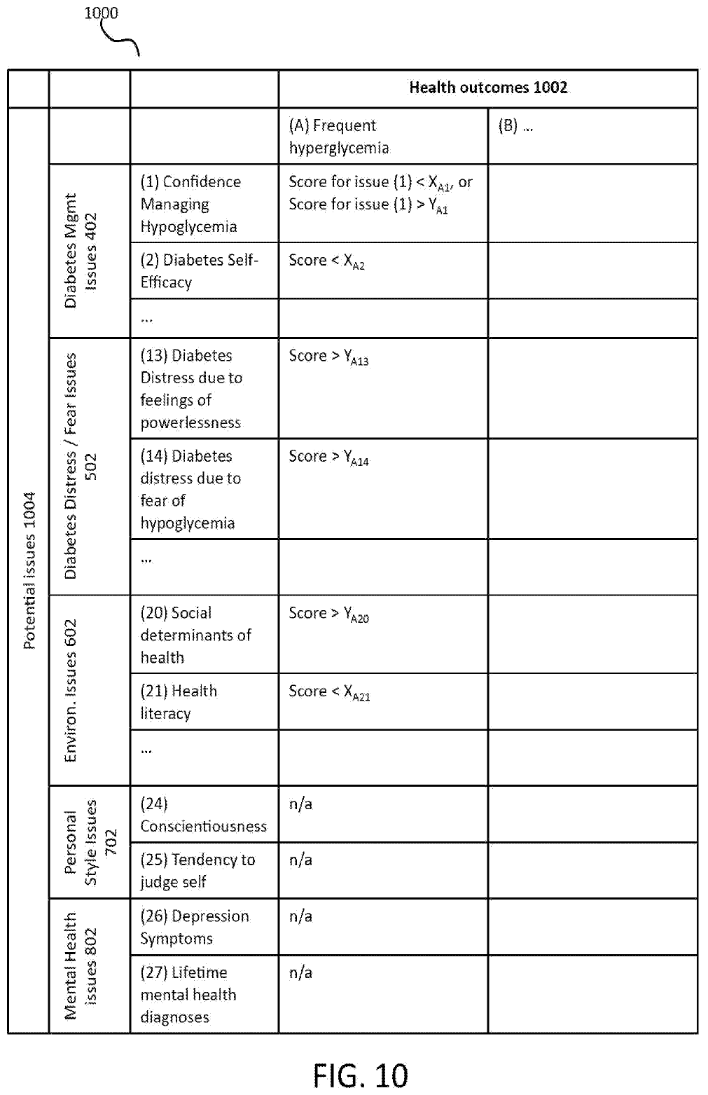

FIG. 10 depicts a table 1000 that illustrates exemplary logic for generating behavioral insights executed by insight generation logic 1508 (see FIG. 15). The parameters X and Y presented in table 1000 are configurable parameters that may be tuned for different applications. It should be understood that table 1000 is presented as a logical aid only, and the rules illustrated therein may be presented in alternate forms. For example, the logic in table 1000 may be represented using a flow-chart, a formula, a decision tree, a series of nested if-then statements, in pseudocode or code, or using other formats. The logic represented by table 1000 may be stored in memory communicably coupled with one or more processors implementing

Each row of table 1000 corresponds to a different potential social, financial, emotional, and/or psychological issue 1004 that the person may be experiencing problems with. As previously described, each issue (labeled (1) through (27)) may be associated with a single score that indicates the extent to which the person is experiencing that corresponding problem. Optionally, the issues (1) through (27) may be further categorized into types of issue categories, e.g., diabetes management issues 402, diabetes distress/fear issues 502, environmental issues 602, personal style issues 702, and/or mental health issues 802. Each column of table 1000 corresponds to a different adverse health outcome 1002. For example, column (A) corresponds to the adverse health outcome of frequent hyperglycemia. Columns (B), (C), etc. correspond to different adverse health outcomes.

For ease of reference, cells within table 1000 shall be referred to by the letter of the column to which it belongs followed by the number of the row to which it belongs. So, for example, cell A1 shall refer to the cell corresponding to adverse health outcome (A) (i.e., “frequent hyperglycemia”) and issue (1) (“Confidence managing hypoglycemia”). Each cell in table 1000 may be populated with criteria for determining whether to generate a behavioral insight that correlates (i) the adverse health outcome corresponding to the column to which that cell belongs with (ii) the social, financial, emotional, and/or psychological issue corresponding to the row to which that cell belongs. One exemplary rule for generating behavioral insights can then be expressed in this way: if the person is experiencing an adverse health outcome associated with column X (where X is a letter), and if the person's issue scores satisfy the criteria in cell XY (where Y is a number), then process 200 generates a behavioral insight correlating the adverse health outcome associated with column X with the issue number Y. So for example, if the person is experiencing an adverse health outcome corresponding to column (A) (i.e., “frequent hyperglycemia”), and if the person's issue scores satisfy the criteria in cell A1 (i.e., “Score for issue (1)<X_{A1}, or Score for issue (1)>Y_{A1}”), then process 200 generates a behavioral insight correlating the adverse health outcome of column (A) “frequent hyperglycemia” with issue (1) (i.e., “Confidence managing hypoglycemia”).

In some embodiments, the criteria for generating a behavioral insight correlating adverse health outcome X with social, financial, emotional, and/or psychological issue Y may include scores that pertain to issues other than issue Y. In the exemplary table 1000, this means that the criteria in cell A1 may, in some instances, evaluate scores associated with issues other than issue (1). This may be the case where the appropriate thresholds to use for issue (1) may vary depending on the scores for another issue. In pseudocode form, the criteria in cell A1 may state: if {(Score for issue (5)>T AND Score for issue (1)>U1) OR (Score for issue (5)<T AND Score for issue (1)>U2)} then generate a behavioral insight comprising a correlation between adverse health outcome (A) and issue (1). In this instance, the threshold for evaluating the score for issue (1) changes between U1 and U2 depending on whether the value of the score for issue (5) is greater than T.

Returning to FIG. 2, at step 214, server 160 generates an indication of the generated behavioral insights, the generated indication adapted to be presented to a user. For example, process 200 may present an indication of the generated behavioral insights to a HCP via a web portal or window within the HCP's Electronic Medical Record (EMR) system when the HCP views record information related to the person. The presented indications alert the HCP to possible correlations, drivers, and/or factors that may exacerbate observed health outcomes detected in the person's diabetes treatment information. These indications allow the HCP to go beyond simply admonishing the person for adverse health outcomes, and to enter a productive conversation with the person regarding possible root causes related to social, financial, emotional, and/or psychological issues behind the health outcomes.

The steps of process 200 may be executed in parallel or in alternative order to that described herein. For example, steps 208-210 may be executed in parallel with or before steps 202-206. Other arrangements of the steps of process 200 are also possible.

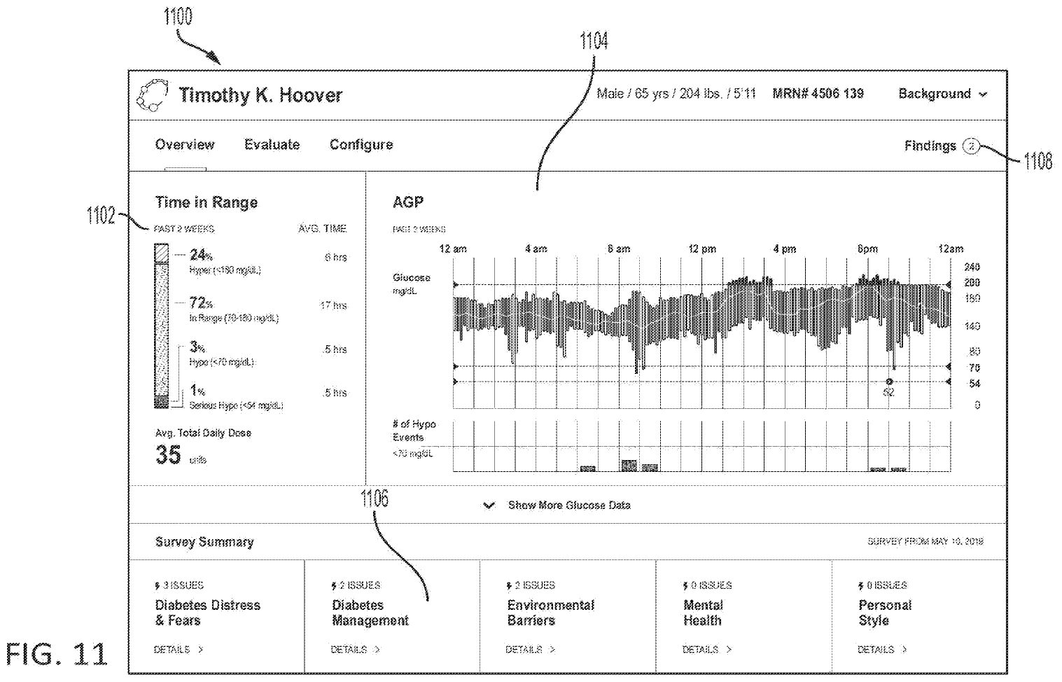

FIGS. 11-14 provide screenshots for an exemplary user-interface for reviewing diabetes treatment information related to a person with diabetes, adverse health outcomes for the person, and behavioral insights generated by process 200. This user-interface may be used by a HCP providing treatment or advice for a person with diabetes.

FIG. 11 is a screenshot 1100 of the exemplary user-interface for reviewing diabetes-related information for a person with diabetes, Timothy K. Hoover. Screen 1100 contains a first panel 1102 that presents summary statistics for the person's glucose levels over a monitored period (in this example, over the “Past 2 weeks”). These summary statistics can include, for example, the proportion of time the person's glucose levels were in-range (70-180 mg/dL), above range (i.e., hyperglycemia, >180 mg/dL), below-range (i.e., hypoglycemia, <70 mg/dL), or seriously below-range (i.e., serious hypoglycemia, <54 mg/dL). The screen 1100 also contains the average number of units of insulin that the person administered per day during the monitored time period. Screen 1100 further contains a panel 1104 that presents an ambulatory glucose profile (AGP) for the person's glucose levels during the monitored period.

Screen 1100 further displays a panel 1106 that highlights for the user social, financial, emotional, and/or psychological problems that the person may be experiencing, based on the person's PRO survey instrument results and scores generated from such results. In this example, panel 1106 contains a sub-panel for each of the different categories of issues, i.e., “Diabetes Management”, “Diabetes Distress & Fears”, “Environmental Barriers”, “Mental Health”, and “Personal Style”. Each sub-panel indicates the number of significant issues that the person may be experiencing within that corresponding category of issues. For instance, according to scores generated from the person's PRO survey instrument responses, the person presented in screen 1100 may be suffering from three problems related to “Diabetes Distress & Fears”, two problems related to “Diabetes Management”, and two problems related to “Environmental Barriers.” Panel 1106 indicates the date on which the PRO survey instruments were taken (in this example: May 10, 2019). An issue may be flagged as “significant”, and therefore worthy of being displayed in panel 1106, if the person's scores associated with that issue satisfy certain criteria or thresholds, e.g., if the person's scores are greater than a threshold, less than a threshold, within a target range, or outside of a target range.

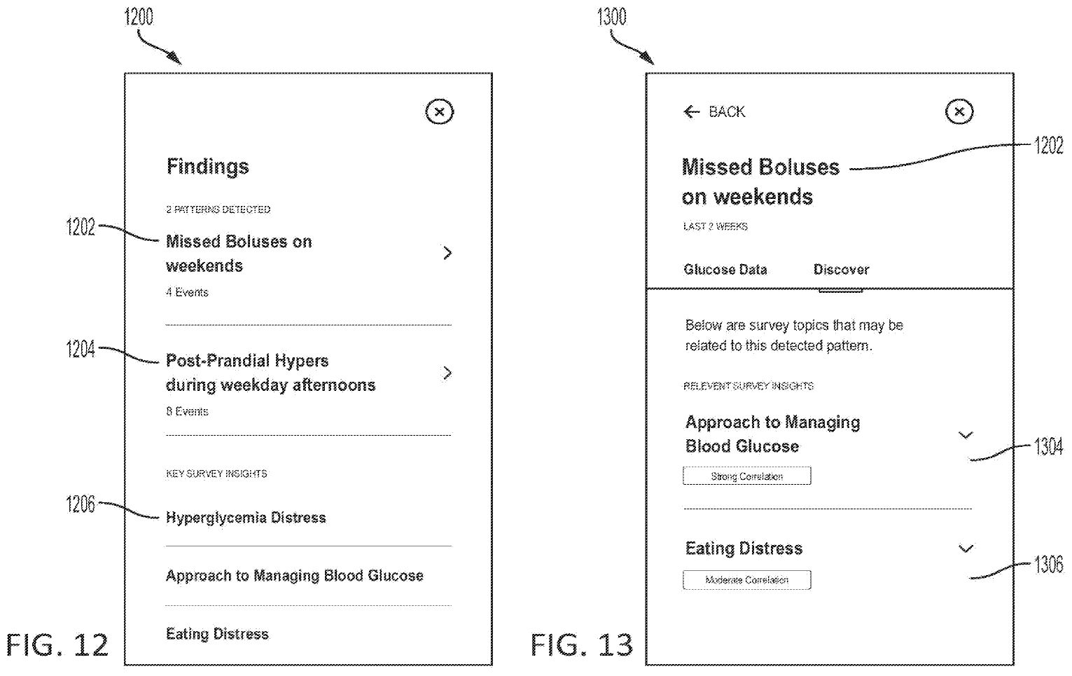

Screen 1100 further includes a “Findings” button 1108. Clicking on the Findings button 1108 opens the sub-panel 1200, depicted in FIG. 12. Sub-panel 1200 displays adverse health outcomes detected in the person's diabetes treatment information. In this example, sub-panel 1200 displays two detected health outcomes: outcome 1202 associated with missed boluses on weekends, and outcome 1204 associated with post-prandial hypers during weekday afternoons. Each outcome is associated with a number of “events”, e.g., 4 events for outcome 1202 and 8 events for outcome 1204. These “events” indicate the number of occurrences of that respective health outcome during the monitored time period. Sub-panel 1200 further includes a section 1206 that displays social, financial, emotional, and/or psychological issues that were surfaced by the person's PRO survey instrument responses and generated scores, and which may be related to some or all of the detected health outcomes.

When the user clicks on one of the displayed detected health outcomes, e.g., outcome 1202 associated with missed boluses on weekends, the user-interface may display the sub-panel 1300, depicted in FIG. 13. Sub-panel may provide further detail regarding a single health outcome, in this case, outcome 1202 associated with missed boluses on weekends. If the user clicks on the tab for “Glucose data”, sub-panel 1300 displays glucose information related to those detected health outcomes. If the user clicks on the tab “Discover”, sub-panel 1300 displays social, financial, emotional, and/or psychological issues that were surfaced by the person's PRO survey instrument responses and generated scores, and which may be correlated with the health outcome “missed boluses on weekends.” In this example, sub-panel 1300 displays two issues: issue 1304 related to Approach to Managing Blood Glucose, and issue 1306 related to Eating Distress. In this way, sub-panels 1200 and 1300 display to the user the generated behavioral insights as potential factors that may impact health outcomes for the person.

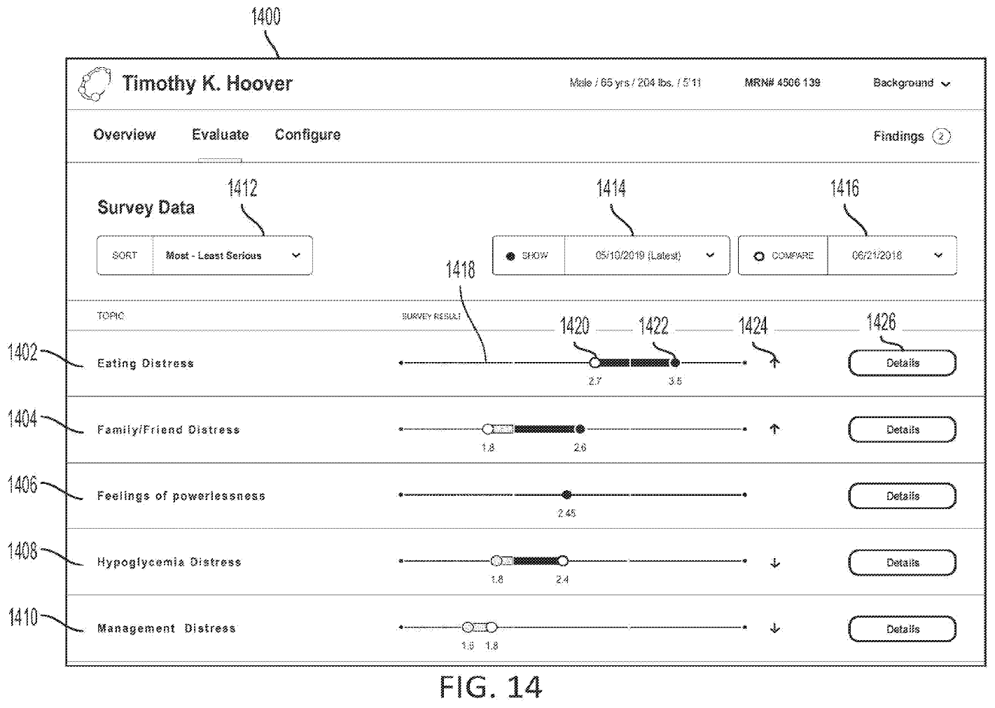

When the user clicks on either of the displayed issues 1304 and/or 1306, the user is taken to the screen 1400 depicted in FIG. 14. Screen 1400 displays the person's scores associated with different social, financial, emotional, and/or psychological issue 1402, 1404, 1406, 1408, and 1410. By manipulating the drop-down box 1412, the user may select how to sort the displayed issues, e.g., from Most-Least Serious, or from Least-Most Serious. By manipulating the drop-down box 1414, the user may select the date of the PRO survey instrument to be viewed. By manipulating the drop-down box 1416, the user may select the date of the PRO survey instruments against which the current results should be compared.

Screen 1400 may display a score range line 1418 for each issue 1402, 1404, 1406, 1408, and 1410. The range line pictorially depicts where the person's scores for each issue fall on a spectrum, from least serious on the left to most serious on the right. A current score marker 1422 indicates where the person's current scores (i.e., the scores generated from PRO survey instruments responses received on the date selected in drop-down box 1414) fall on this range-line. A previous score marker 1420 indicates where the person's previous scores (i.e., the scores generated from PRO survey instrument responses received on the date selected in the drop-down box 1416) fall on this range-line. In this way, the user can quickly see not only where the person's current score on a particular issue falls, but also compare with the person's previous score on this issue to see whether the patient's score is improving or getting worse. A trend indicator 1424 also pictorially indicates whether the person's score is improving or getting worse—an up arrow indicates the person's score is going up or getting worse, while a down arrow indicates the person's score is going down or getting better. Clicking on a button 1426 associated with one of the issues will display the person's actual responses to the PRO survey instrument related to that issue.

FIG. 15 is a block diagram that illustrates the logical components within server 160 for implementing process 200, according to some embodiments. As shown, processing circuit 162 of server 160 may implement at least four different types of logic: Patient Reported Outcome (PRO) scheduling logic 1502, PRO scoring logic 1504, health outcome analysis logic 1506, and insight generation logic 1508. As described previously, each type of logic may take the form of software and/or firmware stored in non-transitory computer-readable media (such as memory 164) executed in processing circuit 162 to implement the functions described herein.

PRO scheduling logic 1502 may comprise logic configured to send, via the communication device 166 and the network 150, an electronic invitation to complete one or more patient reported outcome (PRO) survey instruments to a device associated with the person with diabetes, the PRO survey instruments configured to measure at least one of the person's social, financial, emotional, and psychological state. PRO scheduling logic 1502 may also determine the appropriate time to send such electronic invitations. For example, PRO scheduling logic 1502 may be configured to send the electronic invitation on a regularly scheduled periodic basis, such as once every six months or once a year. PRO scheduling logic 1502 may alter the periodic frequency at which invitations are sent based on different factors, such as based on user-input (e.g., from a HCP or from the person with diabetes), or when scores generated from the person's previous PRO survey instrument responses indicate the person requires more or less frequent monitoring. PRO scheduling logic 1502 may also send the invitations at random intervals within certain parameters. PRO scheduling logic 1502 may also send the electronic invitation at ad hoc, unscheduled times based on user-input, such as upon request by a HCP or by the person with diabetes.

PRO scoring logic 1504 may comprise logic configured to receive, via the communication device and the network, electronic responses to the one or more PRO survey instruments from the device associated with the person. The logic 1504 may also be configured to score the responses to generate one or more scores associated with the person according to the methods and processes disclosed herein, wherein each score of the one or more scores is indicative of the extent to which the person is experiencing a different social, financial, emotional, or psychological problem. The logic 1504 may also be configured to store at least one of the responses and the generated one or more scores in the memory.

Health outcome analysis logic 1506 may comprise logic configured to receive, via the communication device and the network, diabetes treatment information associated with the person, the diabetes treatment information including at least one of insulin dosage information collected by a connected insulin delivery device and glucose measurement information collected by a connected glucose measurement device. Logic 1506 may also be configured to analyze the diabetes treatment information to derive one or more adverse health outcomes experienced by the person, according to the methods and processes disclosed herein.

Insight generation logic 1508 may comprise logic configured to generate one or more behavioral insights according to the methods and processes disclosed herein. Each behavioral insight comprises a correlation between one of the derived adverse health outcomes with one or more of the generated scores associated with the person. Logic 1508 also presents to the user an indication of the generated behavioral insights as potential factors that may impact health outcomes for the person, according to the methods and processes disclosed herein.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

Various aspects are described in this disclosure, which include, but are not limited to, the following aspects:

1. A computerized method for generating and presenting to a user behavioral insights impacting health outcomes for a person with diabetes, the method comprising: sending, by one or more processors to a device associated with the person with diabetes, an electronic invitation via a network to execute one or more electronic patient reported outcome (PRO) survey instruments, each PRO survey instrument configured to measure at least one a social state, a financial state, an emotional state, and a psychological state of the person; receiving, at the one or more processors via the network, at least one electronic response to the one or more PRO survey instruments from the device associated with the person; scoring, by the one or more processors, the at least one electronic response to generate one or more scores associated with the person, wherein each score of the one or more scores is indicative of the extent to which the person is experiencing a different social, financial, emotional, or psychological issue; receiving, by the one or more processors via the network, diabetes treatment information for the person collected over a monitored time period, the diabetes treatment information including at least one of insulin dosage information and glucose measurement information; analyzing, by the one or more processors, the diabetes treatment information to derive one or more adverse health outcomes experienced by the person during the monitored time period; automatically generating, by the one or more processors, one or more behavioral insights, wherein each behavioral insight comprises a correlation between one of the derived adverse health outcomes with one or more of the generated scores associated with the person; and generating an indication of the one or more behavioral insights, the generated indication adapted to be presented to the user.

2. The method of aspect 1, wherein the diabetes treatment information includes at least one of insulin dosage information collected by a connected insulin delivery device and glucose measurement information collected by a connected glucose measurement device.

3. The method of any of aspects 1-2, wherein generating the one or more behavioral insights comprises, for each respective adverse health outcome experienced by the person: providing a set of issues associated with the respective adverse health outcome, the set of issues including at least one of a social issue, a financial issue, an emotional issue, and a psychological issue; providing one or more score criteria for each issue in the set of issues; comparing the one or more generated scores associated with the person to the one or more score thresholds to determine a subset of issues within the set of issues, wherein the one or more generated scores satisfies the one or more score criteria for each issue in the subset of issues; and generating a separate behavioral insight of the one or more behavioral insights for each issue in the subset of issues.

4. The method of aspect 3, wherein the provided sets of social, financial, emotional, or psychological issues and the provided sets of one or more score criteria are stored in memory communicably coupled with the one or more processors in the form of a decision tree, look-up table, formula, or code.

5. The method of any of aspects 1-4, wherein the one or more adverse health outcomes comprises at least one of episodes of hypoglycemia and hyperglycemia.

6. The method of any of aspects 1-5, wherein the one or more adverse health outcomes comprises at least one of a high variation in glucose levels, and insufficient time-in-range.

7. The method of any of aspects 1-6, wherein the one or more adverse health outcomes comprises at least one of a missed bolus, a late bolus, an insufficient bolus, an excessive bolus, an improper upward dose override, and an improper downward dose override.

8. The method of any of aspects 1-7, wherein the one or more generated scores comprises at least one of a score that is indicative of a confidence of the person in managing hypoglycemic episodes, a score is indicative of a level of diabetes self-efficacy, and a score that is indicative of a level of motivation of the person in managing diabetes.

9. The method of any of aspects 1-7, wherein the one or more generated scores comprises a score that is indicative of a confidence of the person in managing hypoglycemic episodes.

10. The method of any of aspects 1-9, wherein the one or more generated scores comprise at least one of a score that is indicative of health literacy of the person, a score that is indicative of a level of conscientiousness of the person, and a score that is indicative of a presence of depression or anxiety symptoms in the person.

11. The method of any of aspects 1-10, wherein the one or more generated scores comprise a score that is indicative of a presence of depression or anxiety symptoms in the person.

12. The method of any of aspects 1-11, wherein the generated indication of the one or more behavioral insights comprises a visual display that: displays one of the derived adverse health outcomes experienced by the person; and for each respective score of the one or more generated scores that are correlated with the displayed adverse health outcome by the one or more behavioral insights, displays an indication of the social, financial, emotional, or psychological issue indicated by the respective score.

13. A system for generating and presenting to a user behavioral insights impacting health outcomes for a person with diabetes, the system comprising: memory; a communication device communicably coupled to a network; and one or more processors configured to execute instructions stored in the memory to implement: patient reported outcome (PRO) scheduling logic that is configured to send, via the communication device and the network to a device associated with the person with diabetes, an electronic invitation to execute one or more electronic patient reported outcome (PRO) survey instruments, the PRO survey instruments configured to measure at least one of a social state, a financial state, an emotional state, and a psychological state of the person; PRO scoring logic that is configured to: receive, via the communication device and the network, at least one electronic response to the one or more PRO survey instruments from the device associated with the person, score the at least one electronic response to generate one or more scores associated with the person, wherein each score of the one or more scores is indicative of the extent to which the person is experiencing a social, financial, emotional, or psychological problem, and store at least one of the responses and the generated one or more scores in the memory; health outcome analysis logic that is configured to: receive, via the communication device and the network, diabetes treatment information associated with the person, the diabetes treatment information including at least one of insulin dosage information and glucose measurement information, and analyze the diabetes treatment information to derive one or more adverse health outcomes experienced by the person; insight generation logic that is configured to: generate one or more behavioral insights, wherein each behavioral insight comprises a correlation between one of the derived adverse health outcomes with one or more of the generated scores associated with the person, and generate an indication of the one or more behavioral insights, the generated indication adapted to be presented to the user.

14. The system of aspect 13, wherein the diabetes treatment information includes at least one of insulin dosage information collected by a connected insulin delivery device and glucose measurement information collected by a connected glucose measurement device.

15. The system of any of aspects 13-14, wherein the health outcome analysis logic is configured to, for each respective adverse health outcome experienced by the person: provide a set of issues associated with the respective adverse health outcome, the set of issues including at least one of a social issue, a financial issue, an emotional issue, and a psychological issue; provide one or more score criteria for each issue in the set of issues; compare the one or more generated scores associated with the person to the one or more score criteria to determine a subset of issues within the set of issues, wherein the one or more generated scores satisfy the one or more score criteria for each issue in the subset of issues; and generate a separate behavioral insight of the one or more behavioral insights for each issue in the subset of issues.

16. The system of aspect 15, wherein the provided sets of social, financial, emotional, or psychological issues and the provided sets of one or more score criteria are stored in memory communicably coupled with the one or more processors in the form of a decision tree, look-up table, formula, or code.

17. The system of any of aspects 13-16, wherein the one or more adverse health outcomes comprises at least one of episodes of hypoglycemia and hyperglycemia.

18. The system of any of aspects 13-17, wherein the one or more adverse health outcomes comprises at least one of a high variation in glucose levels, and insufficient time-in-range.

19. The system of any of aspects 13-18, wherein the one or more adverse health outcomes comprises at least one of a missed bolus, a late bolus, an insufficient bolus, an excessive bolus, an improper upward dose override, and an improper downward dose override.

20. The system of any of aspects 13-19, wherein the one or more generated scores comprises at least one of a score that is indicative of a confidence of the person in managing hypoglycemic episodes, a score that is indicative of a level of diabetes self-efficacy, and a score that is indicative of a level of motivation of the person in managing diabetes.

21. The system of any of aspects 13-20, wherein the one or more generated scores comprises a score that is indicative of a confidence of the person in managing hypoglycemic episodes.

22. The system of any of aspects 13-21, wherein the one or more generated scores comprise at least one of a score that is indicative of health literacy of the person, a score that is indicative of a level of conscientiousness of the person, and a score that is indicative of a presence of depression or anxiety symptoms in the person.

23. The system of any of aspects 13-22, wherein the one or more generated scores comprise a score that is indicative of a presence of depression or anxiety symptoms in the person.

24. The system of any of aspects 13-23, wherein the generated indication of the one or more behavioral insights comprises a visual display that: displays one of the derived adverse health outcomes experienced by the person; and for each respective score of the one or more generated scores that are correlated with the displayed adverse health outcome by the one or more behavioral insights, displays an indication of the social, financial, emotional, or psychological issue indicated by the respective score.

25. Non-transitory computer-readable media storing computer-executable instructions that, when executed by one or more processors, are operable to cause the one or more processors to: send an electronic invitation via a network to execute one or more electronic patient reported outcome (PRO) survey instruments to a device associated with the person with diabetes, the PRO survey instruments configured to measure at least one of a social state, a financial state, an emotional state, and a psychological state of the person; receive, via the network, at least one electronic response to the one or more PRO survey instruments from the device associated with the person; score the at least one electronic response to generate one or more scores associated with the person, wherein each score of the one or more scores is indicative of the extent to which the person is experiencing a social, financial, emotional, or psychological issue; receive, via the network, diabetes treatment information for the person collected over a monitored time period, the diabetes treatment information including at least one of insulin dosage information and glucose measurement information; analyze the diabetes treatment information to derive one or more adverse health outcomes experienced by the person during the monitored time period; automatically generate one or more behavioral insights, wherein each behavioral insight comprises a correlation between one of the derived adverse health outcomes with one or more of the generated scores associated with the person; and generate an indication of the one or more behavioral insights, the generated indication adapted to be presented to the user.

26. The non-transitory computer-readable media of aspect 25, wherein the diabetes treatment information includes at least one of insulin dosage information collected by a connected insulin delivery device and glucose measurement information collected by a connected glucose measurement device.

27. The non-transitory computer-readable media of any of aspects 25-26, wherein generating the one or more behavioral insights comprises, for each respective adverse health outcome experienced by the person: providing a set of issues associated with the respective adverse health outcome, the set of issues including at least one of a social issue, a financial issue, an emotional issue, and a psychological issue; providing one or more score criteria for each issue in the set of issues; comparing the one or more generated scores associated with the person to the one or more score criteria to determine a subset of issues within the set of issues, wherein the one or more generated scores satisfies the one or more score criteria for each issue in the subset of issues; and generating a separate behavioral insight of the one or more behavioral insights for each issue in the subset of issues.

28. The non-transitory computer-readable media of aspect 27, wherein the provided sets of social, financial, emotional, or psychological issues and the provided sets of one or more score thresholds are stored in the non-transitory computer-readable media in the form of a decision tree, look-up table, formula, or code.

29. The non-transitory computer-readable media of any of aspects 25-28, wherein the one or more adverse health outcomes comprises at least one of episodes of hypoglycemia and hyperglycemia.

30. The non-transitory computer-readable media of any of aspects 25-29, wherein the one or more adverse health outcomes comprises at least one of a high variation in glucose levels, and insufficient time-in-range.

31. The non-transitory computer-readable media of any of aspects 25-30, wherein the one or more adverse health outcomes comprises at least one of a missed bolus, a late bolus, an insufficient bolus, an excessive bolus, an improper upward dose override, and an improper downward dose override.

32. The non-transitory computer-readable media of any of aspects 25-31, wherein the one or more generated scores comprises at least one of a score that is indicative of a confidence of the person in managing hypoglycemic episodes, a score that is indicative of a level of diabetes self-efficacy, and a score that is indicative of a level of motivation of the person in managing diabetes.

33. The non-transitory computer-readable media of any of aspects 25-32, wherein the one or more generated scores comprises a score that is indicative of a confidence of the person in managing hypoglycemic episodes.

34. The non-transitory computer-readable media of any of aspects 25-33, wherein the one or more generated scores comprise at least one of a score that is indicative of health literacy of the person, a score that is indicative of a level of conscientiousness of the person, and a score that is indicative of a presence of depression or anxiety symptoms in the person.

35. The non-transitory computer-readable media of any of aspects 25-34, wherein the one or more generated scores comprise a score that is indicative of a presence of depression or anxiety symptoms in the person.

36. The non-transitory computer-readable media of any of aspects 25-35, wherein the generated indication of the one or more behavioral insights comprises a visual display that: displays one of the derived adverse health outcomes experienced by the person; and for each respective score of the one or more generated scores that are correlated with the displayed adverse health outcome by the one or more behavioral insights, displays an indication of the social, financial, emotional, or psychological issue indicated by the respective score.

1. computerized method for generating and presenting to a user behavioral insights impacting health outcomes for a person with diabetes, the method comprising:
sending, by one or more processors to a device associated with the person with diabetes, an electronic invitation via a network to execute one or more electronic patient reported outcome (PRO) survey instruments, each PRO survey instrument configured to measure at least one a social state, a financial state, an emotional state, and a psychological state of the person;
receiving, at the one or more processors via the network, at least one electronic response to the one or more PRO survey instruments from the device associated with the person;
scoring, by the one or more processors, the at least one electronic response to generate one or more scores associated with the person, wherein each score of the one or more scores is indicative of an extent to which the person is experiencing a different social, financial, emotional, or psychological issue;
receiving, by the one or more processors via the network, diabetes treatment information for the person collected over a monitored time period, the diabetes treatment information including at least one of insulin dosage information and glucose measurement information;
analyzing, by the one or more processors, the diabetes treatment information to derive one or more adverse health outcomes experienced by the person during the monitored time period;
automatically generating, by the one or more processors, one or more behavioral insights, wherein each behavioral insight comprises a correlation between one of the derived adverse health outcomes with one or more of the generated scores associated with the person; and
generating an indication of the one or more behavioral insights, the generated indication adapted to be presented to the user.

2. The method of claim 1, wherein the diabetes treatment information includes at least one of insulin dosage information collected by a connected insulin delivery device and glucose measurement information collected by a connected glucose measurement device.

3. The method of claim 1, wherein generating the one or more behavioral insights comprises, for each respective adverse health outcome experienced by the person:
providing a set of issues associated with the respective adverse health outcome, the set of issues including at least one of a social issue, a financial issue, an emotional issue, and a psychological issue;
providing one or more score criteria for each issue in the set of issues;
comparing the one or more generated scores associated with the person to the one or more score criteria to determine a subset of issues within the set of issues, wherein the one or more generated scores satisfies the one or more score criteria for each issue in the subset of issues; and
generating a separate behavioral insight of the one or more behavioral insights for each issue in the subset of issues.

4. The method of claim 3, wherein the provided sets of social, financial, emotional, or psychological issues and the provided sets of one or more score criteria are stored in memory communicably coupled with the one or more processors in the form of a decision tree, look-up table, formula, or code.

5. The method of claim 1, wherein the one or more adverse health outcomes comprises at least one of episodes of hypoglycemia and hyperglycemia.

6. The method of claim 1, wherein the one or more adverse health outcomes comprises at least one of a high variation in glucose levels, and insufficient time-in-range.

7. The method of claim 1, wherein the one or more adverse health outcomes comprises at least one of a missed bolus, a late bolus, an insufficient bolus, an excessive bolus, an improper upward dose override, and an improper downward dose override.

8. The method of claim 1, wherein the one or more generated scores comprises at least one of a score that is indicative of a confidence of the person in managing hypoglycemic episodes, a score that is indicative of a level of diabetes self-efficacy, and a score that is indicative of a level of motivation of the person in managing diabetes.

9. The method of claim 1, wherein the one or more generated scores comprises a score that is indicative of a confidence of the person in managing hypoglycemic episodes.

10. The method of claim 1, wherein the one or more generated scores comprise at least one of a score that is indicative of health literacy of the person, a score that is indicative of a level of conscientiousness of the person, and a score that is indicative of a presence of depression or anxiety symptoms in the person.

11. The method of claim 1, wherein the one or more generated scores comprise a score that is indicative of a presence of depression or anxiety symptoms in the person.

12. The method of claim 1, wherein the generated indication of the one or more behavioral insights comprises a visual display that:
displays one of the derived adverse health outcomes experienced by the person; and
for each respective score of the one or more generated scores that are correlated with the displayed adverse health outcome by the one or more behavioral insights, displays an indication of the social, financial, emotional, or psychological issue indicated by the respective score.

13. A system for generating and presenting to a user behavioral insights impacting health outcomes for a person with diabetes, the system comprising:
memory;
a communication device communicably coupled to a network; and
one or more processors configured to execute instructions stored in the memory to implement:
patient reported outcome (PRO) scheduling logic that is configured to send, via the communication device and the network to a device associated with the person with diabetes, an electronic invitation to execute one or more electronic patient reported outcome (PRO) survey instruments, the PRO survey instruments configured to measure at least one of a social state, a financial state, an emotional state, and a psychological state of the person;
PRO scoring logic that is configured to:
receive, via the communication device and the network, at least one electronic response to the one or more PRO survey instruments from the device associated with the person,
score the at least one electronic response to generate one or more scores associated with the person, wherein each score of the one or more scores is indicative of an extent to which the person is experiencing a social, financial, emotional, or psychological problem, and
store at least one of the responses and the generated one or more scores in the memory;
health outcome analysis logic that is configured to:
receive, via the communication device and the network, diabetes treatment information associated with the person, the diabetes treatment information including at least one of insulin dosage information and glucose measurement information, and
analyze the diabetes treatment information to derive one or more adverse health outcomes experienced by the person;
insight generation logic that is configured to:
generate one or more behavioral insights, wherein each behavioral insight comprises a correlation between one of the derived adverse health outcomes with one or more of the generated scores associated with the person, and
generate an indication of the one or more behavioral insights, the generated indication adapted to be presented to the user.

14. The system of claim 13, wherein the diabetes treatment information includes at least one of insulin dosage information collected by a connected insulin delivery device and glucose measurement information collected by a connected glucose measurement device.

15. The system of claim 13, wherein the health outcome analysis logic is configured to, for each respective adverse health outcome experienced by the person:
provide a set of issues associated with the respective adverse health outcome, the set of issues including at least one of a social issue, a financial issue, an emotional issue, and a psychological issue;
provide one or more score criteria for each issue in the set of issues;
compare the one or more generated scores associated with the person to the one or more score criteria to determine a subset of issues within the set of issues, wherein the one or more generated scores satisfy the one or more score criteria for each issue in the subset of issues; and
generate a separate behavioral insight of the one or more behavioral insights for each issue in the subset of issues.

16. The system of claim 15, wherein the provided sets of social, financial, emotional, or psychological issues and the provided sets of one or more score criteria are stored in memory communicably coupled with the one or more processors in the form of a decision tree, look-up table, formula, or code.

17. The system of claim 13, wherein the one or more adverse health outcomes comprises at least one of episodes of hypoglycemia and hyperglycemia.

18. The system of claim 13, wherein the one or more adverse health outcomes comprises at least one of a high variation in glucose levels, and insufficient time-in-range.

19. The system of claim 13, wherein the one or more adverse health outcomes comprises at least one of a missed bolus, a late bolus, an insufficient bolus, an excessive bolus, an improper upward dose override, and an improper downward dose override.

20. The system of claim 13, wherein the one or more generated scores comprises at least one of a score that is indicative of a confidence of the person in managing hypoglycemic episodes, a score that is indicative of a level of diabetes self-efficacy, and a score that is indicative of a level of motivation of the person in managing diabetes.

21. The system of claim 13, wherein the one or more generated scores comprises a score that is indicative of a confidence of the person in managing hypoglycemic episodes.

22. The system of claim 13, wherein the one or more generated scores comprise at least one of a score that is indicative of health literacy of the person, a score that is indicative of a level of conscientiousness of the person, and a score that is indicative of a presence of depression or anxiety symptoms in the person.

23. The system of 13, wherein the one or more generated scores comprise a score that is indicative of a presence of depression or anxiety symptoms in the person.

24. The system of claim 13, wherein the generated indication of the one or more behavioral insights comprises a visual display that:
displays one of the derived adverse health outcomes experienced by the person; and
for each respective score of the one or more generated scores that are correlated with the displayed adverse health outcome by the one or more behavioral insights, displays an indication of the social, financial, emotional, or psychological issue indicated by the respective score.

25. Non-transitory computer-readable media storing computer-executable instructions that, when executed by one or more processors, are operable to cause the one or more processors to:
send an electronic invitation via a network to execute one or more electronic patient reported outcome (PRO) survey instruments to a device associated with a person with diabetes, the PRO survey instruments configured to measure at least one of a social state, a financial state, an emotional state, and a psychological state of the person;
receive, via the network, at least one electronic response to the one or more PRO survey instruments from the device associated with the person;
score the at least one electronic response to generate one or more scores associated with the person, wherein each score of the one or more scores is indicative of an extent to which the person is experiencing a social, financial, emotional, or psychological issue;
receive, via the network, diabetes treatment information for the person collected over a monitored time period, the diabetes treatment information including at least one of insulin dosage information and glucose measurement information;
analyze the diabetes treatment information to derive one or more adverse health outcomes experienced by the person during the monitored time period;
automatically generate one or more behavioral insights, wherein each behavioral insight comprises a correlation between one of the derived adverse health outcomes with one or more of the generated scores associated with the person; and
generate an indication of the one or more behavioral insights, the generated indication adapted to be presented to the user.

26. The non-transitory computer-readable media of claim 25, wherein the diabetes treatment information includes at least one of insulin dosage information collected by a connected insulin delivery device and glucose measurement information collected by a connected glucose measurement device.

27. The non-transitory computer-readable media of claim 25, wherein generating the one or more behavioral insights comprises, for each respective adverse health outcome experienced by the person:
providing a set of issues associated with the respective adverse health outcome, the set of issues including at least one of a social issue, a financial issue, an emotional issue, and a psychological issue;
providing one or more score criteria for each issue in the set of issues;
comparing the one or more generated scores associated with the person to the one or more score criteria to determine a subset of issues within the set of issues, wherein the one or more generated scores satisfies the one or more score criteria for each issue in the subset of issues; and
generating a separate behavioral insight of the one or more behavioral insights for each issue in the subset of issues.

28. The non-transitory computer-readable media of claim 27, wherein the provided sets of social, financial, emotional, or psychological issues and the provided sets of one or more score criteria are stored in the non-transitory computer-readable media in the form of a decision tree, look-up table, formula, or code.

29. The non-transitory computer-readable media of claim 25, wherein the one or more adverse health outcomes comprises at least one of episodes of hypoglycemia and hyperglycemia.

30. The non-transitory computer-readable media of claim 25, wherein the one or more adverse health outcomes comprises at least one of a high variation in glucose levels, and insufficient time-in-range.

31. The non-transitory computer-readable media of claim 25, wherein the one or more adverse health outcomes comprises at least one of a missed bolus, a late bolus, an insufficient bolus, an excessive bolus, an improper upward dose override, and an improper downward dose override.

32. The non-transitory computer-readable media of claim 25, wherein the one or more generated scores comprises at least one of a score that is indicative of a confidence of the person in managing hypoglycemic episodes, a score that is indicative of a level of diabetes self-efficacy, and a score that is indicative of a level of motivation of the person in managing diabetes.

33. The non-transitory computer-readable media of claim 25, wherein the one or more generated scores comprises a score that is indicative of a confidence of the person in managing hypoglycemic episodes.

34. The non-transitory computer-readable media of claim 25, wherein the one or more generated scores comprise at least one of a score that is indicative of health literacy of the person, a score that is indicative of a level of conscientiousness of the person, and a score that is indicative of a presence of depression or anxiety symptoms in the person.

35. The non-transitory computer-readable media of claim 25, wherein the one or more generated scores comprise a score that is indicative of a presence of depression or anxiety symptoms in the person.

36. The non-transitory computer-readable media of claim 25, wherein the generated indication of the one or more behavioral insights comprises a visual display that:
displays one of the derived adverse health outcomes experienced by the person; and
for each respective score of the one or more generated scores that are correlated with the displayed adverse health outcome by the one or more behavioral insights, displays an indication of the social, financial, emotional, or psychological issue indicated by the respective score.
